SEQUENCE LISTING (5 sequences)

SEQ ID NO: 1 - DNA, 12459 bp, Homo sapiens
  repeat_region (98)..(383)
gatatcctattaatacagagatacagaaagaaatacataaaaaatagttttatcaaatactttccagcattcaagtgtagcctcaaaagcaagaataggccaggagtggtggctcacgctgtaatccacagcactgtgggaggccaaggtaagaggattgcttgaggccaggatttcaagaccagcctaggcaacatagtgagatccctatctctacgaaaaaattttaaaacttagctgggcatggtgcttgagcctgttgtcccagctactcaggaggtgaagtaggagtgtcacttgagcccaggaggttgaggctgcagtgagctataactgcaccactgcactccagccttggagacagagtgagaccctgtccccaaaaaaattaaaattgagaaaaaaaaaaaggcaagaacagccacagcaaactttctattggggaaaaaaaaaaatcctcctctttacatctctcccttccttcccttccctttctgagagtgactgtggccaaaaggagcattttccccctgcagtcctctgaggggtggggtggggctatgaagctatccttcatattcactcctttgtccagctcttttcacctctagttcttctccccgcatctctgtctagcagtgccttaagtggaggaggggtgggggcatcaagcttgtaaaactggtttgttggggttctccttctcccctcatttcttgattcttgggaaaatgtcttgctgggaggctgcctggcgagtgccctagctgccttctgtgggcttgaatggggcttccctctgcccctacaggaggaaaagggagctgctgccagagggagaaatggagagatggacagagaaggcaggtgccacccctcgcccctgacacacaaagaaaaagacacggaaattctctctctctcttctcttctcctatctctctctctctccctctctctctctctctctctctctctcacacacacacacacacacacacacacacacacaggcgcgcgccgcgcgcgcaggcacacgtcttgcaaattcaggattcaaagagacaggggcaccattatatttggcacggtggggccttccaggtctgaaatcctgcattcttccttactatttactttccccgagctcgagaagggccaggtgtgggcggatggctggccacgttttgtgtttccaattcatattcacgggatgacacagacggggcgtggtgagtgctgttggaggcgcttgggcagtttcattttgccccacttctccacctgaaggctgggcgttgctggaacctgcaggggcagcctcagcaaggtggggtggcgtggagtggggtgggagaagggactccagctgaagtagaacccaggctggacctgagaatattggggagggcatgggcggtggtttccgggtaggggccttgaggacatgttggtcctgactgttgtcagtgtttggtcaaagttgccaaaaggttaaaaaaaaaaaagtagggggagtccctgccaagacatatttcccaggccacctttcttccgcgggagtgttgggggggaggcgctgcttggaacctgtgaatgtgacatcagctctcctctcctctcccaaggtcggctttggagagggaggtcagggcacccttgcctggcacaggcacgctggcttccggctcagtgccgcctgctctccgggagctgtgcgctccctgggccccggggctaggctgaggtaagcgcacagcggaggccaggcgcgccggcagaggcctgggggatagggtggaggcatctctgggtgtgggtgtgggtgtgggtgtgggagggagagttcttgcctctctctctcccatctccaactcttgcttcagtggctcttttagaggatgcatgtcattatggacctgtcgctgccactgtccctgttcccccagctgtgacttcgagggaggtctggggatctgagtctgtccaaacccacggctttgctgttgggataaaaactgtccttttgattttagaaggaggagggaaaaaaggtttcccagcatgtgtgttgtgccagtcttggaaattcatccgtgcttgaattccaccctccatccccagaaaaactggagtaaaacaaaaagaggagatggacaaagtgtgtatttgatggcatcccctgggaagagactctaaatttatcccataggtcttactgggccactgtgagcgctttggtggagaacaaacaaaaattctgggtgctcagttgtctaacctgaaaaatgggactagcggaaaaagccaatgtgttccatgcaccttttgctttctttattaaggcatgatgtcacctgtacagtaactgccctgtgtgtacttcagggggggatttcaaggttagatagacaggaaattgttttgaaaatgtaaacacattattaaatgtgaagtattatctgattccttgttcgaatggcatttccttctcagcaccaccttccttgcatattcacttaaccttgtacaagaacacctttttgccctaaatgaagacacccccccaaaaaaaagagtcccagaaaatatgtccctgcttgtgcgggaataaatagaatattctgaggtgcattcctccttcctatgttaggcaacattccttgaccctcctcggcccccaagccaggttgcgtttttttctgccatttagaagggttttcctttttgtcctagtaaaacatcagcccctgtagctcttcatctccccctggtgttcttctcccgccatgtcttaagattggtggcaccgaccaatcttaagatttaagttctgtgtgaaaaacacctttgcttttcaatcagtttatcagcctcctccgcaggggaatgtggacacacaaaagaacttatcggggcttctcatcagtgatagggaaaagactggcatgtgcctaaacgagctctgatgttatttttaagctccctttcttgccaatccctcacggatctttctccgatagatgcaaagaacttcagcaaaaaagacccgcaggaaggggcttgaagagaaaagtacgttgatctgccaaaatagtctgacccccagtagtgggcagtgacgagggagagcattcccttgtttgactgagactagaatcggagagacataaaaggaaaatgaagcgagcaacaattaaaaaaaattccccgcacacaacaatacaatctatttaaactgtggctcatacttttcataccaatggtatgactttttttctggagtcccctcttctgattcttgaactccggggctggcagcttgcaaaggggaagcggactccagcactgcacgggcaggtttagcaaaggtctctaatgggtattttctttttcttagccctgcccccgaattgtcagacggcgggcgtctgcttctgaagttagcagtgatttcctttcgggcctggcttatctccggctgcacgttgcctgttggtgactaataacacaataacattgtctggggctggaataaagtcggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacaggtaggcacgctcgttgacttgtaagtctcggaattacaagttagtgtgttcttatccaccttcatgcttttcttgcttctatttttccccgttctttttatgactgcagcttagagagcaagtgtctgagaattattgctgaaacgtactttaagtcttctagtgtaaaatgtaaaattcctctactgaatacaattaggtgcaattgactataacatgacattaaaataacttatcgttttattattattattccattatgtgtttccttggcttttaaaaaatgagaagagtatggacatatacaatttagtcaaatgtatgtttgtaatatatgtgtttatacaggtacacaggccatataggaacttaaatcttatttaaacactattttaatagtgtgttaacgtgtaaaatatttaagcattccagcttgaagccaaggaattgtatccagtcgttcaagcaatgtatgttcagtaaaatcacctgcagagcaaaagtctgttgactaactaccgcctcccccgcccccccaccaccccccgcaggcggtttctgggtgaagcagatgttttctttaaaatttgtcatcattgactttaggtttcttttggcaggtttttggcacccaaaacagtgtgagctctcttttcagctttattcacctgtgctgggaggggagctaggataattcttggctgccgaaggatttaggcagtgcgtgtgcatctgcccgggtcccccccgtttttagggtcagtgcactttttttgtcttttcgtgaccctgactaaagagaaaggatgtcaagggaatgaaaatcctggaatgtgtctgatcatttgaaatgtacaaaattgggcagataagctgcatggctaaattgttaggaggaagaggcaaggcagtagtggagaagggggaggcagtggatcccacacaagcctgatgcccagggattcggaattcaaaatccccccagcctaccttcagtcccctgacctgcttctcagccccaccttaggtcactggtttctatggagttaccctactgaattgaatattgaatagttaatttctctctccaatcattttccccacctaattttgaaagatatacatcatctggggtaccctgtgccctacacagcatgtgaagtggatgggtaccccctaaagagagggtcatcctgaatggggaagtggccccaaagctaggaataactgtgatttcttgtctttagtcatgtgccaatgttaagtaagcttcagtggatagtgctgtcctaccaagttccttgtagaagccagccggattttcaacaggcagcattccacagcatttccctgagcctgcttcaagaggggtgggggaagtcccttttcaggtgtttatctcctctgcatttgtgtaatctccctgaaggtggataagccaagggcatgagggggaggcaaaaggtgaactcatgttaaggagggaaaaaaataaagagcccttttttctgtgtttcttgctgatggcaggctgtgtgcttcatctgcttttatctgctctgctagctctgactctactgtgatccagcatgtctctcggcgtttgaggagacatcccccactgacctgctctttctctccccagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagtaagtctctagagggcattgtaaccctattcattcattagcgctggctccactggagcccagttttagagtttcttttctagggactctgaaggtagtccttctaacaccatccaagtgcctcagtggggacagtttccctctattcctgaaaataacgacagcttcgttcttagcaaccaaggggagggtcttctgaggccccgtagctcaggctactcatgatgggacaagcaggaggccactgcacgtttcaaatgaggaactttcagtgagagggcctcagggggacactctcacagtggcatctgatggggtttcgggaataattgccgaggtcagatgtgggttagtgcaacctgtgcttctcatgggagggtggagactgagaggcagaagtgatgatatagagggttagaatcacttaattttagttacagaaaaacctaggctcaaagtgttgaagccatttgtgcaggagtgagtttgtagcagagctagaactggagcccggatttcctttgctgctatattttccctttagaaatgcccatttcagaactgaaatagaaatactgtccataggcttctctttcacctacagagaagaaaagcagatttcctccttctgccctggacactagttcatcatctgtcggaagcagtcataaacaagcacacatttactatgcatacaatgtaccgttatgacaaaggaggaccaaaatccaaacaatatcaaaccacaccaaaaaccacaaggagcctaataattactaaggtgatacttccaaagggaggactttatttcttagatgagaatgaaaatggacacattggaaattattggagagccctctggctatgagtccttccacaaccatatggtaccaccgactggcaggagaaatgtgtgaacatgtgcctcctctccccaaccactggggccggtggggtgacggtggcacttttagcagtatcctccgtggtttgagttgaaaataagttttaaaaatcctgtgagtcatggttttgcattgaaacctcttcccactgtgtacccacaaatagttaactaaatagaccattagaaaaggaagaaaatataaagcagatgccaagcagagatgtcctaatttttgacaaaaaagcaatgttgcttgtgtcaagaagaaactgaactttgtgaagagttgaaatggaattccactgaattagaaaaacttgttttctcctgcctggatacatacagtcagggccattgatgcacaggtgttcctggctgttgttacactttaccctctgaaatgatgctcccaagtgctatgtgatgagctccttgtgtgcccagtggaataggtgtgtccatgtgtcattttaaagactattaattacactaatatagtttctttctctctttggataataggcacgttgttccgtatggacttggaagccctaggtccaagagagccttggagaatttacttcccacaaaggcaacagaccgtgagaatagatgccaatgtgctagccaaaaagacaagaagtgctggaatttttgccaagcaggaaaagaactcaggtgagcagaaacacctttgcttttcaatcagtttaacagcctcctgaactccttcctatcatggtactgccttcctgttttagagagactaacagagacattgaaagtcagggtaaagctgaatataacattgctgaaatgtttttccttgtgtattttaacagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtaagagggaaggaagaaaaattaggtaagaggttcacaagaacaactagccccagtcagtgatgccagcagcctgttcctccagcccttcttacccgggcaggtgaaagacttagaaaacagtagcagaggagatctatgcatcctatagattaaaaggagcaaaagaatccctcttaaatatttccatgaagctctggaatgcaaaccgatgtcctctgtacctttagcacataccatttcatctacaggtagatttcccaaccaaaatatatccagagatgcctttgtcattgggttatatacagcctttgcctctctgagtcaatgtatttaccactttccctgagaaatcgaaaatcattttggggagcggacatttagaaaaagaatcaaagtgtcatggataatcaaattcttcaataagttgcagttattcagatggccaaaggaaaaataaagtcattagatagggttggtagaatttagaacatgctgtttttcaggtttatggtctttttttttttttttttttttttttaaatagggaaatgtgtttggtgcagagccaatgtcattccaaaaagctctctcttttcctggtcagtcatgtgctgggacagagaagggatctggattaggcaacatcatagagttgctctgagctgctctttggtgataacccttccaaatcctaaactttttggaattcacaagctcaaaggaggaaacctactctctgatctaccacatgttctgcatttttctatcatggtctatggaaacttctcttagaaatccagtggcaagaagttctatgattaaagtgttctgagctcaggccaggcagtcatgaactacttctgagttgtttactactgatttgtggggcagcctcagctatcggtttcttcacacctgcttatgagagtatccatatttatggtcgcaggcagtaatgctccccacgagatcagtttctgaactaacctggaattttttatgggtttttattatgccaactattaaatcaacattacagttcttccctctgtatttctcctgtaaaacattaggcctgcaaaaaaaaaaaatctttttaaaaataattgccataaagtatttgctctgggcctactgtatgcttcttttytttttctctcttttcaactaagtcaccgtcaatttattaagatggccataactattcaaaacctatgctgagttcctcaaggcagggtcgcatagtgatgaaggttgggatggggctacggaagaaaccagaacaactctagtttatttaaaacctgtatttactgcccacttccccttagacttgaccatatgaccccttgctccccattctaagcataggggcaggctttatttttacaatggtaatagatgatatcacttgaggttttatcaaagagttgcggcgggtggtgaaagttcacaaccagattcaggttttgtttgtgccagattctaattttacatgtttcttttgccaaagggtgatttttttaaaataacatttgttttctcttatcttgctttattaggtcggagaccatgagaaacagcgtcaaatcatcttttcatgatcccaagctgaaaggcaagccctccagagagcgttatgtgacccacaaccgagcacattggtgacagaccttcggggcctgtctgaagccatagcctccacggagagcctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaagaaagacttttgtttaaatttgtaaaatgcaaaactgaatgaaactgttactaccataaatcaggatatgtttcatgaatatgagtctacctcacctatattgcactctggcagaagtatttcccacatttaattattgcctccccaaactcttcccacccctgctgccccttcctccatcccccatactaaatcctagcctcgtagaagtctggtctaatgtgtcagcagtagatataatattttcatggtaatctactagctctgatccataagaaaaaaaagatcattaaatcaggagattccctgtccttgatttttggagacacaatggtatagggttgtttatgaaatatattgaaaagtaagtgtttgttacgctttaaagcagtaaaattattttcctttatataaccggctaatgaaagaggttggattgaattttgatgtacttatttttttatagatatttatattcaaacaatttattccttatatttaccatgttaaatatctgttgggcaggccatattggtctatgtatttttaaaatatgtatttctaaatgaaattgagaacatgctttgttttgcctgtcaaggtaatgactttagaaaataaatatttttttccttactgtactgatttggaatcattactgaaatttgtaaggagtgggccaacgtgattaagtaccataaaggcaaataaatggttaaagacggtttcatagaaaagtgacaattagaaggatattacggtctaagctaattatataaagaattttatctgtatcttaaatgttgattttatactgcattgaggtaaaaacacaaaacaaaaaagcagctttaacacctctgtcttctcttgggtagcagcctcctgcttctccttcacctgaaaaattctccagggacttcatccattaacttggctcaggctattggcaggattcacagtttaagctgatggtgtggtgagagatgctttatccatattaatggactgaaggaagtaatggcaagacaaccccccaaaacatacctaattatacaaagttatataccaaagttgcttttagaaaatggcctgctcagagcaagtagaggtttccaatggctttttattttctcacattaaggatgttgtttcttaaggaacattgagtaccattgcttcttcgtgatagcctaggactgccgtgtgcccatggaggtagagacaccaggtactgattctaggtcctctgccacaaagcaccacttcctctccactttgccttggctggccttgtcagctcactggagagcacagtattgcaattgcagtattgcaaatggtcactactaactgaattctctaagagcttgattagccctcgagaatcttccttgcccttctctaatagtgtctgaaggaattcctggcatttaacaaatattagcatgtagtgatcactgtcgtcctaacagtgacacatcagaaggatttcaaataacagtcttcaggcatgcgtaatcaatgtcctgtgcagagtctccgtcctcattgatcctcatttttctctttaaggcacagtccaatgtctttggggaattgtttataaagcttactttatccataaactgtttctcagtgcgtgactctgaagaaaattttgaagttttgcccatgttgacaaggtgcttggtctgaacttggccagtatttaatcttgagcaaacgattcaatttccttctatcgtgagttttctcatctatgaaacaagggagttgaggggagtttctttcatacctctgagaaagagtttgagattacataaagaagttgaagtggcatgaaaaaaaataaagatctgagcttagaagacatggatctaatacatttaagaggaagtcagaatcagagaagccactgaacaaaacagtccaaacggagcatagtaagtcagattgatgagttttggttgggtttttcatcagtcaaacccttgagcccccctttcccatgcttcctgcttcagtatccagtaggaaaaatgaaagggatgatgtagacactctagggcatgaggatttgcagtaaataagttgggagactcacagaaaattaatatttttcaaacatgaagacgaaacattcaattatattacagtccacatcagcttgaagggtaaactgatgggatgatctgtcacatttcttgctctgtttccagtaaaagcatggtttctggaaacccacttaggacagctttctctctttacactgatagcccaggcaagctttgatctcagaactccagaaaccagagaactctaggtggaatgtggtaacttttgccagggcagagggaacacctactaataggtacttcatttgcaccaccagagattggcatcttttttgatggatccactggctttgatactgcctgtactcccccaaaacacagcttgggtattggactaatctagagctccctcaggagaactcttgctgacattaagaaagagcaacattttgtctttccaggtgaaaatccaaggccaaaaagggagtgactcacctaagatcacagaaggagctgtagcatctctggagcctgaacacttaagttaagcacgactatttcacgcagagggcatgaattc

SEQ ID NO: 2 - DNA, 20 bp, Artificial sequence
  Primer
ctccatccccagaaaaactg

SEQ ID NO: 3 - DNA, 20 bp, Artificial sequence
  Primer
aaggaaggtggtgctgagaa

SEQ ID NO: 4 - DNA, 20 bp, Artificial sequence
  Primer
gggggatttcaaggttagat

SEQ ID NO: 5 - DNA, 22 bp, Artificial sequence
  Primer
gagaagccccgataagttcttt

(19) United States
(12) Patent Application Publication
(10) Pub. No.: US 2004/0191774 A1
(43) Pub. Date: Sep. 30, 2004

(54) Endothelin-1 promoter polymorphism

(76) Inventor: David W Moskowitz, St Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/380,024

(22) PCT Filed: Sep. 11, 2001
(86) PCT No.: PCT/US01/28834

(51) Int. Cl. C12Q 1/68

(52) U.S. Cl. 435/6

(57) ABSTRACT

Disclosed are single nucleotide polymorphisms (SNPs) associated with hypertension, end stage renal disease due to hypertension non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infarction due to hyper-tension, atherosclerotic peripheral vascular disease due to non-insulin dependent diabetes mellitus, cerebrovascular accident non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder. Also disclosed are methods for using the SNPs to determine susceptibility to these diseases; nucleotide sequences containing the SNPs; kits for determining the presence of the SNPs; and methods of treatment or prophylaxis based on the presence of the SNPs.

### BACKGROUND

This invention relates to detection of individuals at risk for pathological conditions based on the presence of single nucleotide polymorphisms (SNPs) at positions 2239 and 2657 on the human endothelin-1 (EDN-1) promoter.

During the course of evolution, spontaneous mutations appear in the genomes of organisms. It has been estimated that variations in genomic DNA sequences are created continuously at a rate of about 100 new single base changes per individual (Kondrashow, J. Theor. Biol., 175:583-594, 1995; Crow, Exp. Clin. Immunogenet., 12:121-128, 1995). These changes, in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and skis quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome is the single nucleotide polymorphism or SNP. SNPs account for approximately 90% of human DNA polymorphism (Collins et al., Genome Res., 8:1229-1231, 1998). SNPs are single base pair positions in genomic DNA at which different sequence alternatives (alleles) exist in a population. In addition, the least frequent allele must occur at a frequency of 1% or greater. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions (e.g. A→G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998). The frequency of SNPs varies with the type and location of the change. In base substitutions, two-thirds of the substitutions involve the C⇄T (G⇄A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct, as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect, where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP) (U.S. Pat. Nos. 5,324,631; 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases has also been used to detect SNPs.

There is growing recognition that SNPs can provide a powerful tool for the detection of individuals whose genetic make-up alters their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

### SUMMARY

The present inventor has discovered novel single nucleotide polymorphisms (SNPs) associated with the development of various diseases, including hypertension (HTN), end stage renal disease due to hypertension (ESRD due to HTN), non-insulin dependent diabetes mellitus (NIDDM), end stage renal disease due to non-insulin dependent diabetes mellitus (ESRD due to NIDDM), lung cancer, breast cancer, prostate cancer, colon cancer, atherosclerotic peripheral vascular disease due to hypertension (ASPVD due to HTN), cerebrovascular accident due to hypertension (CVA due to HTN), cataracts due to hypertension (cataracts due to HTN), cardiomyopathy with hypertension (HTN CM), myocardial infarction due to hypertension (MI due to HTN), atherosclerotic peripheral vascular disease due to non-insulin dependent diabetes mellitus (ASPVD due to NIDDM), cerebrovascular accident due to non-insulin dependent diabetes mellitus (CVA due to NIDDM), ischemic cardiomyopathy (Ischemic CM), ischemic cardiomyopathy with non-insulin dependent diabetes mellitus (Ischemic CM with NIDDM), myocardial infarction due to non-insulin dependent diabetes mellitus (MI due to NIDDM), atrial fibrillation without valvular disease (afib without valvular disease), alcohol abuse, anxiety, asthma, chronic obstructive pulmonary disease (COPD), cholecystectomy, degenerative joint disease (DJD), end stage renal disease and frequent de-clots (ESRD and frequent de-clots), end stage renal disease due to focal segmental glomerular sclerosis (ESRD due to FSGS), end stage renal disease due to insulin dependent diabetes mellitus (ESRD due to IDDM), or seizure disorder. As such, this polymorphism provides a method for diagnosing a genetic predisposition for the development of these diseases in individuals. Information obtained from the detection of SNPs associated with the development of these diseases is of great value in their treatment and prevention.

Accordingly, one aspect of the present invention provides a method for diagnosing a genetic predisposition for HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder in a subject, comprising obtaining a sample containing at least one polynucleotide from the subject, and analyzing the polynucleotide to detect a genetic polymorphism wherein the presence or absence of said genetic polymorphism is associated with an altered susceptibility to developing HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the polymorphism is located in the EDN-1 gene.

Another aspect of the present invention provides an isolated nucleic acid sequence comprising at least 10 contiguous nucleotides from SEQ ID NO: 1, or their complements, wherein the sequence contains at least one polymorphic site associated with a disease and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention is a kit for the detection of a polymorphism comprising, at a minimum, at least one polynucleotide of at least 10 contiguous nucleotides of SEQ ID NO: 1, or their complements, wherein the polynucleotide contains at least one polymorphic site associated with HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention provides a method for treating HTN, ESRD due to HTN, NIDDM, ESRD due to NIDDM, lung cancer, breast canter, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder comprising, obtaining a sample of biological material containing at least one polynucleotide from the subject; analyzing the polynucleotide to detect the presence of at least one polymorphism associated with HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder; and treating the subject in such a way as to counteract the effect of any such polymorphism detected.

Still another aspect of the invention provides a method for the prophylactic treatment of a subject with a genetic predisposition to HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder comprising, obtaining a sample of biological material containing at least one polynucleotide from the subject; analyzing the polynucleotide to detect the presence of at least one polymorphism associated with HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder, and treating the subject.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### BRIEF DESCRIPTION OF THE DRAWINGS

These and other features, aspects, and advantages of the present invention will become better understood with regard to the following description, appended claims, and accompanying drawings where:

FIG. 1 shows SEQ ID NO: 1, the nucleotide sequence of the EDN-1 promoter region as contained in GenBank Accession Number J05008. 1. Position of the single nucleotide polymorphisms (SNPs) are here given according to the numbering scheme in GenBank Accession Number J05008.1. Thus, all nucleotides will be positively numbered, rather than bear negative numbers reflecting their position upstream from the RNA polymerase II binding site (a TATA box in about half of eukaryotic genes), the transcription initiation site (a variable number of nucleotides downstream of, i.e. 3′ to, the TATA box), the translation start site, or the first codon of the encoded protein (the “A” of the “ATO” codon for methionine, the first amino acid of every protein). Since not all genes are fully annotated, and not all promoter sequences contain elements far downstream such as the “ATG” encoding the first methionine in the translated protein, the numbering system used in this patent application is less troublesome.

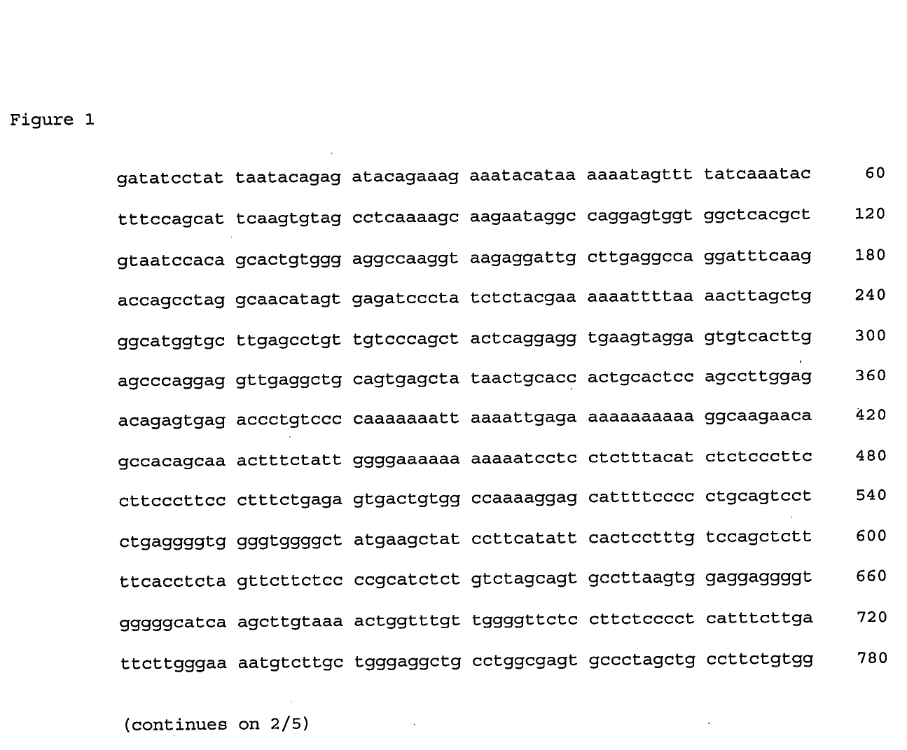

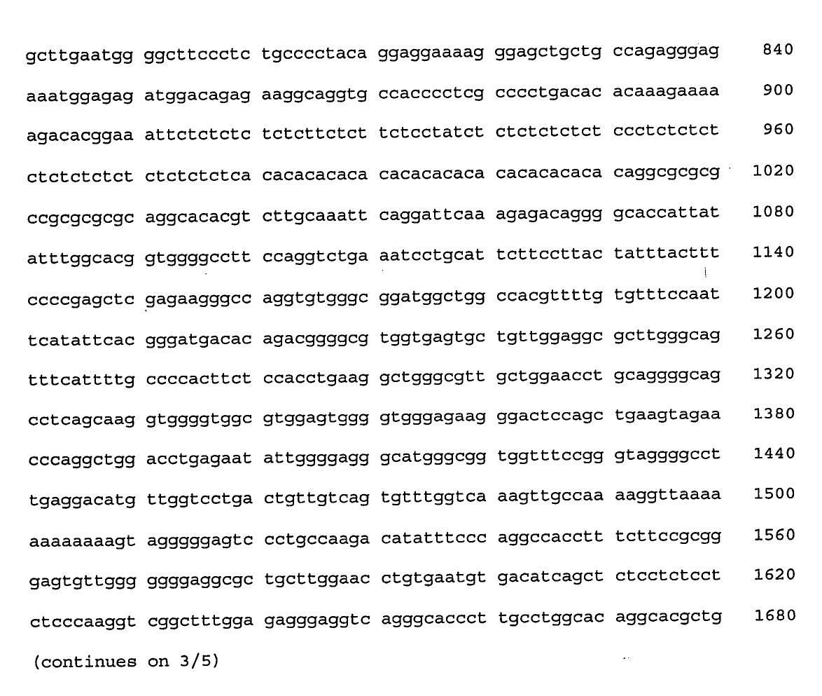

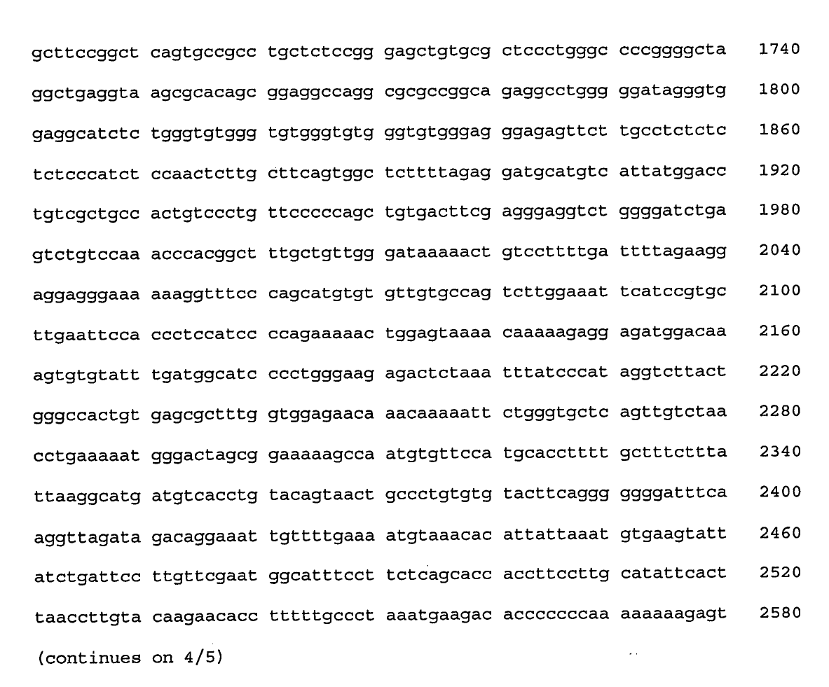

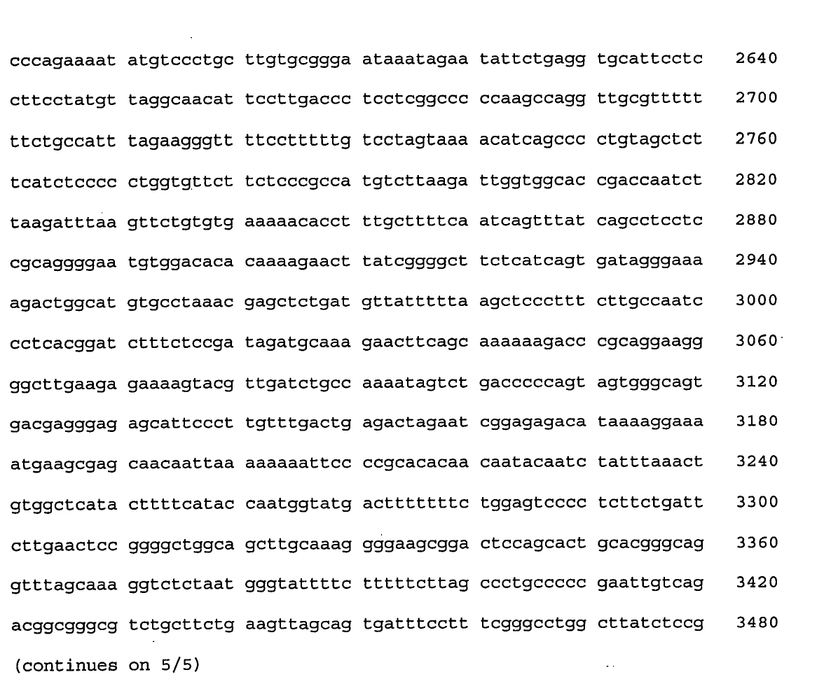

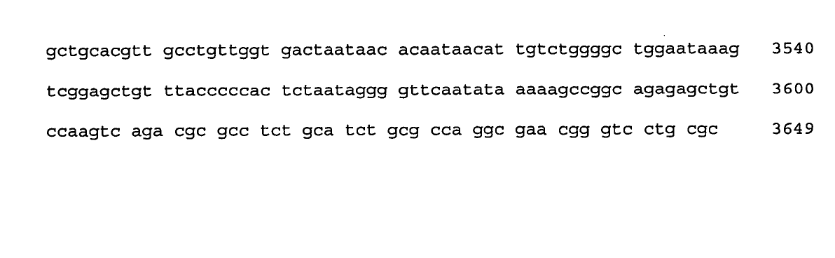

The various numbering systems can be easily interconverted, if desired. According to the annotation of Accession Number, the TATA box is located at position 3577. The first exon begins at position 3608. The position of the ATG codon for the first amino acid (methionine) of the protein is at position 3876.

The first SNP mentioned below is located at position 2239 of Accession Number J05008.1. According to the annotation of Accession Number J05008.1, the transcription start site is position 3608. Therefore, the T2239→G SNP would be −1369 relative to the transcription start position. Further, according to the annotation of Accession Number J05008.1, the position of the “A” of the ATG codon for the first amino acid (methionine) of the protein, i.e.—the translation start site, is at position 3876. The T2239→G SNP corresponds to −1637 with reference to the translation initiation site (the “A” of the first encoded “ATG”).

The second SNP mentioned below (A2657→C) is located at position 2657 according to the numbering scheme of GenBank Accession Number Jp05008.1. Again, according to the annotation of Accession Number J05008.1, the transcription start site is position 3608. Therefore, the A2657→C SNP would be −951 relative to the transcription start position. Further, according to the annotation of Accession Number J05008.1, the position of the “A” of the ATG codon for the first amino acid (methionine) of the protein, i.e.—the translation start site, is at position 3876. The A2657→C SNP corresponds to −1219 with reference to the translation initiation site (the “A” of the first encoded “ATG”).

### DEFINITIONS

nt=nucleotide

bp=base pair

kb=kilobase; 1000 base pairs

ASPVD=atherosclerotic peripheral vascular disease

COPD=chronic obstructive pulmonary disease

CVA=cerebrovascular accident

DJD=degenerative joint disease, also know as osteoarthritis

DOL=dye-labeled oligonucleotide ligation assay

ESRD=end-stage renal disease

FSGS=focal segmental glomerular sclerosis

HTN=hypertension

MASDA=multiplexed allele-specific diagnostic assay

MADGE=microtiter array diagonal gel electrophoresis

MI=myocardial infarction

NIDDM=noninsulin-dependent diabetes mellitus

OLA=oligonucleotide ligation assay

PCR=polymerase chain reaction

RFLP=restriction fragment length polymorphism

SNP=single nucleotide polymorphism

“Polynucleotide” and “oligonucleotide” are used interchangeably and mean a linear polymer of at least 2 nucleotides joined together by phosphodiester bonds and may consist of either ribonucleotides or deoxyribonucleotides.

“Sequence” means the linear order in which monomers occur in a polymer, for example, the order of albino acids in a polypeptide or the order of nucleotides in a polynucleotide.

“Polymorphism” refers to a set of genetic variants at a particular genetic locus among individuals in a population.

“Promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns” between individual coding segments (“exons”). A promoter is herein considered as a part of the corresponding gene. Coding refers to the representation of amino acids, start and stop signals in a three base “triplet” code. Promoters are often upstream (“5′ to”) the transcription initiation site of the gene.

“Gene therapy” means the introduction of a functional gene or genes from some source by any suitable method into a living cell to correct for a genetic defect.

“Reference allele” or “reference type” means the allele designated in the Gen Bank sequence listing for a given gene, in this case Gen Bank Accession Number J05008.1 for the endothelin-1 gene.

“Genetic variant” or “variant” means a specific genetic variant which is present at a particular genetic locus in at least one individual in a population and that differs from the reference type.

As used herein the terms “patient” and “subject” are not limited to human beings, but are intended to include all vertebrate animals in addition to human beings.

As used herein the terms “genetic predisposition”, “genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (reference type) by at least 1%, preferably by at least 2%, more preferably by at least 4% and more preferably still by at least 8%. Altematively, an odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios <1.5).” Id. at 66.

As used herein “isolated nucleic acid” means a species of the invention that is the predominate species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

Novel Polymorphisms

The present application provides single nucleotide polymorphisms (SNPs) in a gene associated with HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. The first polymorphism is a T to G transversion at position 2239 and the second polymorphism is an A to C substitution at position 2657, both of the EDN-1 promoter.

Preparation of Samples

The presence of genetic variants in the above genes or their control regions, or in any other genes that may affect susceptibility to disease is determined by screening nucleic acid sequences from a population of individuals for such variants. The population is preferably comprised of some individuals with the disease of interest, so that any genetic variants that are found can be correlated with disease. The population is also preferably comprised of some individuals that have known risk for the disease. The population should preferably be large enough to have a reasonable chance of finding individuals with the sought-after genetic variant. As the size of the population increases, the ability to find significant correlations between a particular genetic variant and susceptibility to disease also increases.

The nucleic acid sequence can be DNA or RNA. For the assay of genomic DNA, virtually any biological sample containing genomic DNA (e.g., not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In addition, the allele frequency of the genetic variant in a population or subpopulation with the disease or pathology should vary from its allele frequency in the population without the disease or pathology (control population) or the control sequence (reference type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

RFLP analysis (see, e.g. U.S. Pat. Nos. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e., low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled reference type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al., Trends Genet.7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine (T) when mismatched with cytosine, guanine or thymine and the chemical reactivity of cytosine (C) when mismatched with thymine, adenine or cytosine (Cotton et al., Proc. Natl. Acad. Sci. USA, 85:4397-4401, 1988). Duplex DNA formed by hybridization of a reference type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the reference type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998, and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASOs that hybridize to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end, such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons [symbol] method for real time detection of the presence of target sequences or can be used after amplification.

High throughput screening for SNPs that affect restriction sites can be achieved by Microtiter Array Diagonal Gel Electrophoresis (MADGE) (Day and Humphries, Anal. Biochem., 222:389-395, 1994). In this assay restriction fragment digested PCR products are loaded onto stackable horizontal gels with the wells arrayed in a nicrotiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sommer et al., Mayo Clin. Proc., 64:1361-1372, 1989; Sarker et al., Anal. Biochem., 1990), allele-specific amplification (ASA), allele-specific ICR, and amplification refractory mutation system (ARMS) (Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determined whether an individual is homozygous or heterozygous for the mutation (Dutton and Sommer, BioTechlniques, 11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers has a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can be used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled DINP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:346-354, 1996). In this method, incorporation of the ddNTP is determined using an automatic gel sequencer.

Minisequencing has also been adapted for use with microarrals (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled dNTP and noncompeting ddNTPs. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are not suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an increased risk of developing a disease, condition or disorder, and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the genetic variant is at one of the polymorphic sites contained in Table 17. In another embodiment, the genetic variant is one of the variants contained in Table 17 or the complement of any of the variants contained in Table 17. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1, or the complements of SEQ ID NO: 1. In one preferred embodiment, the sequence contains at least one polymorphic site associated with a disease, and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the genetic variant is at one of the polymorphic sites contained in Table 17. In another embodiment, the genetic variant is one of the variants contained in Table 17 or the complement of any of the variants contained in Table 17. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DID, ESRD and frequent de-clots, ESRD due to FSGS, ESRD due to IDDM, or seizure disorder. The kits contain, at a minimum, at least one polynucleotide of at least 10 contiguous nucleotides of SEQ ID NO 1, or the complements of SEQ ID NO: 1. In one embodiment, the genetic variant is at one of the polymorphic sites contained in Table 17. Alternatively the 3′ end of the polynucleotide is immediately 5′ to a polymorphic site, preferably a polymorphic site selected from the sites in Table 17. In another embodiment, the genetic variant is one of the variants contained in Table 17 or the complement of any of the variants contained in Table 17. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder caused either directly or indirectly by the presence of one or more single nucleotide polymorphisms. In this method genetic material from a patient, for example, DNA, cDNA, RNA or mRNA is screened for the presence of one or more SNPs associated with the disease of interest. Depending on the type and location of the SNP, a treatment regime is designed to counteract the effect of the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder, and in particular HTN, ESRD due to HTN, NIDDM, ESRD due to NIDDM, lung cancer, breast cancer, prostate cancer, colon cancer, ASPVD due to HTN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder due to the presence of one or more single nucleotide polymorphisms. In this embodiment, genetic material such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

### EXAMPLES

The positions of the single nucleotide polymorphisms (SNPs) pre given according to the numbering scheme in GenBank Accession Number J05008.1. thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://www.nrbi.nlm.nih.gov/

In the following examples, SNPs are written as “reference sequence nucleotide”→“variant nucleotide.” Changes in nucleotide sequences are indicated in bold print. The standard nucleotide abbreviations are used in which A=adenine, C=cytosine, G=guanine, T=thymine, M=A or C, R=A or G, W=A or T, S=C or G, Y=C or T, K=G or T, V=A or C or G, H=A or C or T; D=A or G or T; B=C or G or T; N=A or C or G or T.

### Example 1

### Detection of Novel Polymorphisms by Direct Sequencing of Leukocyte Genomic DNA

Leukocytes were obtained from human whole blood collected with EDTA as an anticoagulant. Blood was obtained from a group of black men, black women, white men, and white women without any known disease. Blood was also obtained from individuals with HTN, ESRD due to HTN, NIDDM, ESRD due to NIDDM, lung cancer, breast cancer, prostate cancer, colon cancer, ASPVD due to HEN, CVA due to HTN, cataracts due to HTN, HTN CM, MI due to HTN, ASPVD due to NIDDM, CVA due to NIDDM, Ischemic CM, Ischemic CM with NIDDM, MI due to NIDDM, afib without valvular disease, alcohol abuse, anxiety, asthma,.COPD, cholecystectomy, DJD, ESRD and frequent de-clots, ESRD due to FSGS, ESRD due to IDDM, or seizure disorder as indicated in the tables below.

Genomic DNA was purified from the collected leukocytes using standard protocols well known to those of ordinary skill in the art of molecular biology (Ausubel et al., Short Protocol in Molecular Biology, 3^{rd} ed., John Wiley and Sons, 1995; Sambrook et al., Molecular Cloning, Cold Spring Harbor Laboratory Press, 1989; and Davis et al., Basic Methods in Molecular Biology, Elsevier Science Publishing, 1986). One hundred nanograms of purified genomic DNA were used in each PCR reaction.

Standard PCR reaction conditions were used. Methods for conducting PCR are well known in the art and can be found, for example, in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

The first SNP T2239→G can be identified by PCR amplification of a specific region of the endothelin-1 promoter. The sequence of the sense primer was 5′-CTC CAT CCC CAG AAA AAC TG-3′, corresponding to nucleotides 2113 to 2132, inclusive. (SEQ ID NO: 2). The sequence of the anti-sense primer is 5′-AAG GAA GGT GGT GCT GAG AA-3′ corresponding to nucleotides 2490 to 2509, inclusive. (SEQ ID NO: 3). The PCR product spanned positions 2113 to 2509, inclusive, of the EDN1 gene.

The second SNP A2657→C can be identified by PCR amplification of a specific region of the endothelin-1 promoter. The sense primer was 5′-GGG GGA TTT CAA GGT TAG AT -3′ (SEQ ID NO: 4). The anti-sense primer was 5′-GAG AAG CCC CGA TAA GTT CTT T-3′ (SEQ ID NO: 5). The PCR product thus produced spanned positions 2390 to 2924 of the human EDN-1 gene (SEQ ID NO: 1).

The PCR reaction contained a total volume of 20 microliters (μl), consisting of 10 μl of a premade PCR reaction mix (Sigma “JumpStart Ready Mix with RED Taq Polymerase”). Primers at 10 μM were diluted to a final concentration of 0.3 μM in the PCR reaction mix. Approximately 25 ng of template leukocyte genomic DNA was used for each PCR amplification. After an initial 5 minutes denaturation at 94° C., 35 cycles were performed consisting of 45 seconds of denaturation at 94° C., 45 seconds of hybridization at 62° C., 45 seconds of extension at 72° C., followed by a final extension step of 10 minutes at 72° C.

Post-PCR clean-up was performed as follows. PCR reactions were cleaned to remove unwanted primer and other impurities such as salts, enzymes, and unincorporated nucleotides that could inhibit sequencing. One of the following clean-up kits was used: Qiaquick-96 PCR Purification Kit (Qiagen) or Multiscreen-PCR Plates (Millipore, discussed below).

When using the Qiaquick protocol, PCR samples were added to the 96-well Qiaquick silica-gel membrane plate and a chaotropic salt, supplied as “PB Buffer,” was then added to each well. The PB Buffer caused the DNA to bind to the membrane. The plate was put onto the Qiagen vacuum manifold and vacuum was applied to the plate in order to pull sample and PB Buffer through the membrane. The filtrate was discarded. Next, the samples were washed twice using “PE Buffer.” Vacuum pressure was applied between each step to remove the buffer. Filtrate was similarly discarded after each wash. After the last PE Buffer wash, maximum vacuum pressure was applied to the membrane plate to generate maximum airflow through the membrane in order to evaporate residual ethanol left from the PE Buffer. The clean PCR product was then eluted from the filter using EB Buffer.” The filtrate contained the cleaned PCR product ad was collected. All buffers were supplied as part of the Qiaquick-96 PCR-Purification Kit. The vacuum manifold was also purchased from Qiagen for exclusive use with the Qiaquick-96 Purification Kit.

When using the Millipore Multiscreen-PCR Plates, PCR samples were loaded into the wells of the Multiscreen-PCR Plate and the plate was then placed on a Millipore vacuum manifold. Vacuum pressure was applied for 10 minutes, and the filtrate was discarded. The plate was then removed from the vacuum manifold and 100 μl of Milli-Q water was added to each well to rehydrate the DNA samples. After shaking on a plate shaker for 5 minutes, the plate was replaced on the manifold and vacuum pressure was applied for 5 minutes. The filtrate was again discarded. The plate was removed and 60 μl Milli-Q water was added to each well to again rehydrate the DNA samples. After shaking on a plate shaker for 10 minutes, the 60 μl of cleaned PCR product was transferred from the Multiscreen-PCR plate to another 96-well plate by pipetting. The Millipore vacuum manifold was purchased from Millipore for exclusive use with the Multiscreen-PCR plates.

Cycle sequencing was performed on the clean PCR product using an ABI Prism Big Dye Terminator Cycle Sequencing Ready Reaction kit (Perkin-Elmer). For a total volume of 20 μl, the following reagents were added to each well of a 96-well plate: 2.0 μl Terminator Ready Reaction mix, 3.0 μl 5× Sequencing Buffer (ABI), 5-10 μl template (30-90 ng double stranded DNA), 3.2 pM primer (primer used was the forward primer from the PCR reaction), and Milli-Q water to 20 μl total volume. The reaction plate was placed into a Hybaid thermal cycler block and programmed as follows: ×1 cycle: 1 degree/sec thermal ramp to 94° C., 94° C. for 1 min; ×35 cycles: 1 degree/sec thermal ramp to 94° C., then 94° C. for 10 sec, followed by 1 degree/sec thermal ramp to 50° C., then 50° C. for 10 sec, followed by 1 degree/sec thermal ramp to 60° C., then 60° C. for 4 minutes.

The cycle sequencing reaction product was cleaned up to remove the unincorporated dye-labeled terminators that can obscure data at the beginning of the sequence. A precipitation protocol was used. To each sequencing reaction in the 96-well plate, 20 μl of Milli-Q water and 60 μl of 100% isopropanol was added. The plate was left at room temperature for at least 20 minutes to precipitate the extension products. The plate was spun in a plate centrifuge (Jouan) at 3,000×g for 30 minutes.

Without disturbing the pellet, the supernatant was discarded by inverting the plate onto several paper tissues (Kimwipes) folded to the size of the plate. The inverted plate, with Kimwipes in place, was placed into the centrifuge (Jouan) and spun at 700×g for 1 minute. The Kimwipes were discarded and the samples were loaded onto a sequencing gel.

Approximately 1 μi of sequencing product was loaded into each well of a 96-lane 5% Long Ranger (FMC single pack) gel. The running buffer consisted of 1×TBE. The glass plates consisted of ABI 48-cm plates for use with a 96-lane 0.4 mm Mylar shark-tooth comb. A semi-automated ABI Prism 377-96 DNA sequencer was used (ABI 377 with 96-lane, Big Dye upgrades). Sequencing run settings were as follows: run module 48E-1200, 8 hr collection time, 2400 V electrophoresis voltage, 50 mA electrophoresis current, 200 W electrophoresis power, CCD offset of 0, gel temperature of 51° C., 40 mW laser power, and CCD gain of 2.

Pyrosequencing is another method of sequencing DNA by synthesis, where the addition of one of the four dNTPs that correctly matches the complementary base on the template strand is detected. Detection occurs via utilization of the pyrophosphate molecules liberated upon base addition to the elongating synthetic strand. The pyrophosphate molecules are used to make ATP, which in turn drives the emission of photons in a luciferin/luciferase reaction, and these photons are detected by the instrument. A Luc96 Pyrosequencer was used under default operating conditions supplied by the manufacturer. Primers were designed to anneal within 5 bases of the polymorphism, to serve as sequencing primers.

Patient genomic DNA was subject to PCR using amplifying primers that amplify an approximately 200 base pair amplicon containing the polymorphisms of interest. One of the amplifying primers, whose orientation is opposite to the sequencing primer, was biotinylated. This allowed selection of single stranded template for pyrosequencing, whose orientation is complementary to the sequencing primer. Amplicons prepared from genomic DNA were isolated by binding to streptavidin-coated magnetic beads. After denaturation in NaOH, the biotinylated strands were separated from their complementary strands using magnetics. After washing the magnetic beads, the biotinylated template strands still bound to the beads were transferred into 96-well plates. The sequencing primers were added, annealing was carried out at 95° for 2 minutes, and plates were placed in the Pyrosequencer. The enzymes, substrates and dNTPs used for synthesis and pyrophosphate detection were added to the instrument immediately for to sequencing.

The Luc96 software requires definition of a program of adding the four dNTPs that is specific for the location of the sequencing primer, the DNA composition flanking the SNP, and the two possible alleles at the polymorphic locus. This order of adding the bases generates theoretical outcomes of light intensity patterns for each of the two possible homozygous states and the single heterozygous state. The Luc96 software then compares the actual outcome to the theoretical outcome and calls a genotype for each well. Each sample is also assigned one of three confidence scores: pass, uncertain, fail. The results for each plate are output as a text file and processed in Excel using a Visual Basic program to generate a report of genotype and allele frequencies for the various disease and population cell groupings represented on the 96 well plate.

Prediction of potential transcription binding factor sites was performed using a commercially available software program [GENOMATIX MatInspector Professional; URL: http://genomatix.gsf.de/cgi-bin/matinspector/matinspector.pl ; Quandt et al., Nucleic Acids Res., 23: 4878-4884 (1995)].

TABLE 1
GROUP I ALLELE FREQUENCY

| | |
|---|---|
| T | G |

CONTROL

| | | | | |
|---|---|---|---|---|
| Black men (n = 22 chromosomes) | 13 | (59%) | 9 | (41%) |
| Black women (n = 22 chromosomes) | 11 | (50%) | 11 | (50%) |
| White men (n = 18 chromosomes) | 16 | (89%) | 2 | (11%) |
| White women (n = 24 chromosomes) | 21 | (88%) | 3 | (13%) |

DISEASE
BREAST CANCER

| | | | | |
|---|---|---|---|---|
| Black women (n = 24 chromosomes) | 12 | (50%) | 12 | (50%) |
| White women (n = 22 chromosomes) | 19 | (86%) | 3 | (14%) |

LUNG CANCER

| | | | | |
|---|---|---|---|---|
| Black men (n = 24 chromosomes) | 17 | (71%) | 7 | (29%) |
| Black women (n = 4 chromosomes) | 1 | (25%) | 3 | (75%) |
| White men (n = 22 chromosomes) | 15 | (68%) | 7 | (32%) |
| White women (n = 20 chromosomes) | 14 | (70%) | 6 | (30%) |

PROSTATE CANCER

| | | | | |
|---|---|---|---|---|
| Black men (n = 24 chromosomes) | 13 | (54%) | 11 | (46%) |
| White men (n = 24 chromosomes) | 17 | (71%) | 7 | (29%) |

NIDDM

| | | | | |
|---|---|---|---|---|
| Black men (n = 20 chromosomes) | 14 | (70%) | 6 | (30%) |
| Black women (n = 20 chromosomes) | 14 | (70%) | 6 | (30%) |
| White men (n = 22 chromosomes) | 16 | (73%) | 6 | (27%) |
| White women (n = 20 chromosomes) | 13 | (65%) | 7 | (35%) |

ESRD due to NIDDM

| | | | | |
|---|---|---|---|---|
| Black men (n = 8 chromosomes) | 6 | (75%) | 2 | (25%) |
| Black women (n = 20 chromosomes) | 14 | (70%) | 6 | (30%) |
| White men (n = 20 chromosomes) | 18 | (90%) | 2 | (10%) |
| White women (n = 16 chromosomes) | 16 | (100%) | 0 | (0%) |

TABLE 2
GROUP II ALLELE FREQUENCY

| Disease | Race | CHROMOSOMES | N | T | N | G |
|---|---|---|---|---|---|---|
| Controls | African-American | 90 | 61 | 67.8% | 29 | 32.2% |
| | Caucasian | 88 | 76 | 86.4% | 12 | 13.6% |
| Colon cancer | African-American | 44 | 31 | 70.5% | 13 | 29.5% |
| | Caucasian | 44 | 35 | 79.5% | 9 | 20.5% |
| Lung cancer | African-American | 40 | 26 | 65.0% | 14 | 35.0% |
| | Caucasian | 44 | 31 | 70.5% | 13 | 29.5% |
| Hypertension | African-American | 48 | 31 | 64.6% | 17 | 35.4% |
| | Caucasian | 44 | 40 | 90.9% | 4 | 9.1% |
| CVA due to HTN | Caucasian | 46 | 38 | 82.6% | 8 | 17.4% |
| ESRD due to HTN | African-American | 42 | 26 | 61.9% | 16 | 38.1% |
| | Caucasian | 48 | 38 | 79.2% | 10 | 20.8% |
| HTN CM | African-American | 48 | 30 | 62.5% | 18 | 37.5% |
| | Caucasian | 46 | 38 | 82.6% | 8 | 17.4% |
| NIDDM | African-American | 42 | 32 | 76.2% | 10 | 23.8% |
| ASPVD due to NIDDM | Caucasian | 46 | 38 | 82.6% | 8 | 17.4% |
| CVA due to NIDDM | Caucasian | 44 | 39 | 88.6% | 5 | 11.4% |
| ESRD due to NIDDM | Caucasian | 46 | 35 | 76.1% | 11 | 23.9% |
| Ischemic CM with NIDDM | African-American | 48 | 30 | 62.5% | 18 | 37.5% |
| | Caucasian | 48 | 42 | 87.5% | 6 | 12.5% |
| MI due to NIDDM | Caucasian | 48 | 37 | 77.1% | 11 | 22.9% |
| Afib without valvular disease | African-American | 48 | 29 | 60.4% | 19 | 39.6% |
| | Caucasian | 48 | 40 | 83.3% | 8 | 16.7% |
| Alcohol abuse | African-American | 48 | 22 | 45.8% | 26 | 54.2% |
| | Caucasian | 48 | 36 | 75.0% | 12 | 25.0% |
| Asthma | Caucasian | 48 | 41 | 85.4% | 7 | 14.6% |
| COPD | African-American | 40 | 33 | 82.5% | 7 | 17.5% |
| | Caucasian | 42 | 34 | 81.0% | 8 | 19.0% |
| ESRD due to FSGS | Caucasian | 42 | 33 | 78.6% | 9 | 21.4% |

TABLE 3
GROUP I GENOTYPE FREQUENCIES

| | | |
|---|---|---|
| T/T | T/G | G/G |

CONTROLS

| | | | | | | |
|---|---|---|---|---|---|---|
| Black men (n = 11) | 4 | (36%) | 5 | (45%) | 2 | (18%) |
| Black women (n = 11) | 4 | (36%) | 3 | (27%) | 4 | (36%) |
| White men (n = 9) | 8 | (89%) | 0 | (0%) | 1 | (11%) |
| White women (n = 12) | 9 | (75%) | 3 | (25%) | 0 | (0%) |

DISEASE
BREAST CANCER

| | | | | | | |
|---|---|---|---|---|---|---|
| Black women (n = 12) | 4 | (33%) | 4 | (33%) | 4 | (33%) |
| White women (n = 11) | 8 | (73%) | 3 | (27%) | 0 | (0%) |

LUNG CANCER

| | | | | | | |
|---|---|---|---|---|---|---|
| Black men (n = 12) | 6 | (50%) | 5 | (42%) | 1 | (8%) |
| Black women (n = 2) | 0 | (0%) | 1 | (50%) | 1 | (50%) |
| White men (n = 11) | 5 | (45%) | 5 | (45%) | 1 | (9%) |
| White women (n = 10) | 5 | (50%) | 4 | (40%) | 1 | (10%) |

PROSTATE CANCER

| | | | | | | |
|---|---|---|---|---|---|---|
| Black men (n = 12) | 3 | (25%) | 7 | (58%) | 2 | (17%) |
| White men (n = 12) | 5 | (42%) | 7 | (58%) | 0 | (0%) |

NIDDM

| | | | | | | |
|---|---|---|---|---|---|---|
| Black men (n = 10) | 6 | (60%) | 2 | (20%) | 2 | (20%) |
| Black women (n = 10) | 5 | (50%) | 4 | (40%) | 1 | (10%) |
| White men (n = 11) | 7 | (64%) | 2 | (18%) | 2 | (18%) |
| White women (n = 10) | 5 | (50%) | 3 | (30%) | 2 | (20%) |

ESRD due to NIDDM

| | | | | | | |
|---|---|---|---|---|---|---|
| Black men (n = 4) | 2 | (50%) | 2 | (50%) | 0 | (0%) |
| Black women (n = 10) | 5 | (50%) | 4 | (40%) | 1 | (10%) |
| White men (n = 10) | 8 | (80%) | 2 | (20%) | 0 | (0%) |
| White women (n = 8) | 8 | (100%) | 0 | (0%) | 0 | (0%) |

TABLE 4
GROUP II GENOTYPE FREQUENCIES

| Disease | Race | People | N | T/T | N | T/G | N | G/G |
|---|---|---|---|---|---|---|---|---|
| Controls | African-American | 45 | 17 | 37.8% | 27 | 60.0% | 1 | 2.2% |
| | Caucasian | 44 | 33 | 75.0% | 10 | 22.7% | 1 | 2.3% |
| Colon cancer | African-American | 22 | 10 | 45.5% | 11 | 50.0% | 1 | 4.5% |
| | Caucasian | 22 | 15 | 68.2% | 5 | 22.7% | 2 | 9.1% |
| Hypertension | African-American | 24 | 10 | 41.7% | 11 | 45.8% | 3 | 12.5% |
| | Caucasian | 22 | 18 | 81.8% | 4 | 18.2% | 0 | 0.0% |
| CVA due to HTN | Caucasian | 23 | 16 | 69.6% | 6 | 26.1% | 1 | 4.3% |
| ESRD due to HTN | African-American | 21 | 9 | 42.9% | 8 | 38.1% | 4 | 19.0% |
| | Caucasian | 24 | 14 | 58.3% | 10 | 41.7% | 0 | 0.0% |
| HTN CM | African-American | 24 | 10 | 41.7% | 10 | 41.7% | 4 | 16.7% |
| | Caucasian | 23 | 16 | 69.6% | 6 | 26.1% | 1 | 4.3% |
| NIDDM | African-American | 21 | 14 | 66.7% | 4 | 19.0% | 3 | 14.3% |
| ASPVD due to NIDDM | Caucasian | 23 | 16 | 69.6% | 6 | 26.1% | 1 | 4.3% |
| CVA due to NIDDM | Caucasian | 22 | 17 | 77.3% | 5 | 22.7% | 0 | 0.0% |
| ESRD due to NIDDM | Caucasian | 23 | 14 | 60.9% | 7 | 30.4% | 2 | 8.7% |
| Ischemic CM with NIDDM | African-American | 24 | 10 | 41.7% | 10 | 41.7% | 4 | 16.7% |
| | Caucasian | 24 | 18 | 75.0% | 6 | 25.0% | 0 | 0.0% |
| MI due to NIDDM | Caucasian | 24 | 13 | 54.2% | 11 | 45.8% | 0 | 0.0% |
| Afib without valvular disease | African-American | 24 | 9 | 37.5% | 11 | 45.8% | 4 | 16.7% |
| | Caucasian | 24 | 16 | 66.7% | 8 | 33.3% | 0 | 0.0% |
| Alcohol abuse | African-American | 24 | 7 | 29.2% | 8 | 33.3% | 9 | 37.5% |
| | Caucasian | 24 | 14 | 58.3% | 8 | 33.3% | 2 | 8.3% |
| Asthma | Caucasian | 24 | 17 | 70.8% | 7 | 29.2% | 0 | 0.0% |
| COPD | African-American | 20 | 13 | 65.0% | 7 | 35.0% | 0 | 0.0% |
| | Caucasian | 21 | 14 | 66.7% | 6 | 28.6% | 1 | 4.8% |
| ESRD due to FSGS | Caucasian | 21 | 12 | 57.1% | 9 | 42.9% | 0 | 0.0% |

ALLELE-SPECIFIC ODDS RATIOS

The susceptibility allele is indicated below, as well as the odds ratio (OR). The allele which is present more often in the given disease category was chosen as the susceptibility allele. For example, the G allele was chosen as the susceptibility allele for black women with breast cancer because more of the individuals in that category had the G allele than had the T allele. Where there was a “0” in a cell which produced a 0 in the denominator, Haldane's correction (multiplying all cells by 2 and adding 1) was used. If the odds ratio (OR) was ≧1.5, the 95% confidence interval (C.I.) is also given.

An odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios <1.5).” Id. at 66.

An example of the allele-specific odds ratio calculation is given below:

Lung Cancer: Black men

| | Cases | Controls |
|---|---|---|
| T | 17 | 13 |
| G | 7 | 9 |

The odds ratio for the T allele is (17)(9)/(7)(13)=1.7. Therefore, black men with the T allele have a 1.7 fold higher risk of developing lung cancer than black men without the T allele. Odds ratios of 1.5 or higher are highlighted below.

TABLE 5
GROUP I ALLELE-SPECIFIC ODDS RATIOS

| | SUSCEPTIBILITY | | |
|---|---|---|---|
| DISEASE | ALLELE | OR | 95% C.I. |
| Breast Cancer | | | |
| Black women | G | 1.0 | |
| White women | T | 0.9 | |
| Lung Cancer | | | |
| Black men | T | 1.7 | 0.5-5.7 |
| Black women | G | 3.0 | 0.3-33.5 |
| White men | G | 3.7 | 0.7-20.9 |
| White women | G | 3.0 | 0.6-14.0 |
| Prostate Cancer | | | |
| Black men | G | 1.3 | |
| White men | G | 3.3 | 0.6-18.3 |
| NIDDM | | | |
| Black men | T | 1.6 | 0.4-5.8 |
| Black women | T | 2.3 | 0.7-8.3 |
| White men | G | 3.0 | 0.5-17.2 |
| White women | G | 3.8 | 0.8-17.2 |
| ESRD due to NIDDM | | | |
| Black men | T | 1.3* | |
| Black women | T | 1.0* | |
| White men | T | 3.4* | 0.6-19 |
| White women | T | 18.3* | 2.3-148 |

*as compared to NIDDM.

TABLE 6
GROUP II ALLELE-SPECIFIC ODDS RATIOS

| | | | | Lower | Upper | |
|---|---|---|---|---|---|---|
| | | Risk | Odds | Limit | Limit | |
| Disease | Race | Allele | Ratio | 95% CI | 95% CI | Haldane |
| Colon cancer | Caucasian | C | 1.6 | 0.6 | 4.2 | |
| Hypertension | Caucasian | A | 1.6 | 0.5 | 5.2 | |
| CVA due to HTN* | Caucasian | C | 2.1 | 0.6 | 7.6 | |
| ESRD due to HTN* | African-American | C | 1.1 | 0.5 | 2.6 | |
| | Caucasian | C | 2.6 | 0.8 | 9.1 | |
| Ischemic CM with NIDDM*¹ | Caucasian | A | 2.1 | 0.7 | 6.2 | |
| Afib without valvular disease | Caucasian | C | 1.3 | 0.5 | 3.4 | |
| Alcohol abuse | Caucasian | C | 2.1 | 0.9 | 5.2 | |
| Asthma | Caucasian | C | 1.1 | 0.4 | 3.0 | |
| COPD | Caucasian | C | 1.5 | 0.6 | 4.0 | |
| ESRD due to FSGS | Caucasian | C | 1.7 | 0.7 | 4.5 | |

Genotype-Specific Odds Ratios

The susceptibility allele (S) is indicated; the alternative allele at this locus is defined as the protective allele (P). Also presented is the odds ratio (OR) for each genotype (SS, SP; the odds ratio for the PP genotype is 1, since it is the reference group, and is not presented separately). For odds ratios ≧1.5, the 95% confidence interval (C.I.) is also given, in parentheses. Where there was a “0” in a cell which produced a 0 in the denominator, Haldane's correction (multiplying all cells by 2 and adding 1) was used. As discussed above, an odds ratio of 1.5 is chosen as the threshold of significance based on the recommendation of Austin H et al. (Epidemiol. Rev. 16:65-76, 1994).

An example of an odds ratio calculation is worked below, assuming that T is the susceptibility allele (S), and G is the protective allele (P).

Black men: Lung Cancer

| | Cases | Controls | Odds Ratio |
|---|---|---|---|
| TT(SS) | 6 | 4 | (6)(2)/(1)(4) = 3.0 |
| TG(SP) | 5 | 5 | (5)(2)/(1)(5) = 2.0 |
| GG(PP) | 1 | 2 | 1.0 (by definition) |

The odds ratios for individual genotypes are given below. Odds ratios of 1.5 or higher are high-lighted below.

TABLE 7
GROUP I GENOTYPE-SPECIFIC ODDS RATIOS

| | | | |
|---|---|---|---|
| | SUSCEPTI- | | |
| | BILITY | | |
| DISEASE | ALLELE | OR(SS) | OR(SP) |

Lung Cancer

| | | | | | |
|---|---|---|---|---|---|
| Black men | T | 3.0 | (0.2-45.2) | 2.0 | (0.1-29.8) |
| Black women | G | 3.0 | (0.3-34.6) | 3.9 | (0.3-45.6) |
| White men | G | 1.5 | (0.3-9.1) | 17.0 | (1.9-151) |
| White women | G | 5.2 | (0.5-56.1) | 2.2 | (0.6-7.6) |

Prostate Cancer

| | | | | |
|---|---|---|---|---|
| White men | G | 0.5 | 23.2 | (2.7-201) |

NIDDM

| | | | | | |
|---|---|---|---|---|---|
| Black men | T | 1.5 | (0.1-15.5) | 0.4 | |
| Black women | T | 5.0 | (0.4-64.4) | 5.3 | (0.4-75.8) |
| White men | G | 1.9 | (0.4-9.3) | 5.7 | (0.6-54.1) |
| White women | G | 8.6 | (0.9-83.8) | 1.7 | (0.5-6.2) |

ESRD due
to NIDDM

| | | | | | |
|---|---|---|---|---|---|
| White men | T | 5.7 | (0.6-54.1)* | 5.0 | (0.4-59.7)* |
| White women | T | 7.7 | (0.8-75.3)* | 0.7* | |

* - as compared to NIDDM

TABLE 8
GROUP II GENOTYPE-SPECIFIC ODDS RATIOS

| | | RISK | SS | | SP | |
|---|---|---|---|---|---|---|
| Disease | Race | ALLELE | O.R. | HALDANE | O.R. | HALDANE |
| Colon cancer | Caucasian | C | 0.2 | | 0.3 | |
| Hypertension | Caucasian | A | 1.7 | H | 1.3 | H |
| CVA due to HTN* | Caucasian | C | 0.0 | | 0.0 | |
| ESRD due to HTN* | African-American | C | 0.7 | | 0.5 | |
| | Caucasian | C | 0.8 | H | 2.3 | H |
| Ischemic CM with NIDDM*¹ | Caucasian | A | 1.4 | H | 0.6 | H |
| Afib without valvular disease | Caucasian | C | 1.5 | H | 2.4 | H |
| Alcohol abuse | Caucasian | C | 0.2 | | 0.4 | |
| Asthma | Caucasian | C | 1.6 | H | 2.1 | H |
| COPD | Caucasian | C | 0.4 | | 0.6 | |
| ESRD due to FSGS | Caucasian | C | 1.1 | H | 2.7 | H |

*Compared to HTN alone.
*¹Compared to MI with NIDDM.

PCR and sequencing were conducted as described in Example 1. The primers used were the same as in Example 1. The control samples are in good agreement with Hardy-Weinberg equilibrium, as follows:

For the Group I diseases, a frequency of 0.59 for the T allele (“p”) and 0.41 for the G allele (“q”) among black male control individuals predicts genotype frequencies of 35% T/T, 48% T/G, and 17% G/G at Hardy-Weinberg equilibrium (p²+2pq+q²=1). The observed genotype frequencies were 36% T/T, 45% T/G, and 18% C/C, in close agreement with those predicted for Hardy-Weinberg equilibrium.

A frequency of 0.50 for the T allele (“p”) and 0.50 for the G allele (“q”) among black female control individuals predicts genotype frequencies of 25% T/T, 50% T/G, and 25% G/G at Hardy-Weinberg equilibrium (p²+2pq+q²=1). The observed genotype frequencies were 36% T/T, 27% T/G, and 36% C/C, in rather distant agreement with those predicted for Hardy-Weinberg equilibrium.

A frequency of 0.89 for the T allele (“p”) and 0.11 for the G Ilele (“q”) among white male control individuals predicts genotype frequencies of 79% T/T, 20% T/G, and 1% G/G at Hardy-Weinberg equilibrium (p²+2pq+q²=1). The observed genotype frequencies were 89% T/T, 0% T/G, and 11% C/C, in rather distant agreement with those predicted for Hardy-Weinberg equilibrium.

A frequency of 0.88 for the T allele (“p”) and 0.13 for the G allele (“q”) among white female control individuals predicts genotype frequencies of 77% T/T, 21% T/G, and 2% G/G at Hardy-Weinberg equilibrium (p²+2pq+q²=1). The observed genotype frequencies were 75% T/T, 25% T/G, and 0% C/C, in close agreement with those predicted for Hardy-Weinberg equilibrium.

For the Group II diseases, a frequency of 0.68 for the T allele (“p”) and 0.32 for the G allele (“q”,) among African American control individuals predicts genotype frequencies of 45.9% T/T, 44.0% TMG, and 10.1% G/G at Hardy-Weinberg equilibrium (p²+2pq+q²=1). The observed genotype frequencies were 37.8% T/T, 60.0% T/G, and 2.2% G/G, in distant agreement with those predicted for Hardy-Weinberg equilibrium.

A frequency of 0.86 for the T allele (“p”) and 0.14 for the G allele (“q”) among Caucasian control individuals predicts genotype frequencies of 74.6% T/T, 23.5% T/G, and 1.9% G/G at Hardy-Weinberg equilibrium (p²+2pq+q²=1). The observed genotype frequencies were 75.0% T/T, 22.7% T/G, and 2.3% G/G, in excellent agreement with those predicted for Hardy-Weinberg equilibrium.

Using an allele-specific odds ratio of 1.5 or greater as a practical level of significance (see Austin H et al., discussed above), the following observations can be made.

For black men with lung cancer, the odds ratio for the T allele as a risk factor for disease is 1.7 (95% CI, 0.5-5.7). The odds ratio for the homozygote (TT) is 3.0 (95% CI, 0.2- 45.2). The heterozygote (TG genotype) has an odds ratio of 2.0 (95% C.I., 0.1-29.8). These data suggest that the T allele behaves as a dominant allele, with an additive effect of allele dosage (2.0+2.0−1=3.0).

For black women with lung cancer, the odds ratio for the G allele as a risk factor for disease is 3.0 (95% CI, 0.3-33.5). The odds ratio for the homozygote (GG) is 3.0 (95% CI, 0.3-34.6). The heterozygote (GT genotype) has an odds ratio of 3.9 (95% C.I., 0.3-45.6). T hese data suggest that the G allele behaves as a dominant allele, with no additional effect of having two copies of the G allele (GG homozygote) as compared with having only one copy (GT heterozygote).

For white men with lung cancer, the odds ratio for the G allele as a risk factor for disease is 3.7 (95% CI, 0.7-20.9). The odds ratio for the homozygote (GG) is only 1.5 (95% CI, 0.3-9.1), whereas the heterozygote (GT genotype) has a remarkable odds ratio of 17.0 (95% C.I., 1.9-151). These data suggest that the G allele behaves as a codominant allele.

For white women with lung cancer, the odds ratio for the G allele as a risk factor for disease is 3.0 (95% CI, 0.6-14.0). The odds ratio for the homozygote (GG) is 5.2 (95% CI, 0.5-56.1), while the heterozygote (GT genotype) has an odds ratio of 2.2 (95% C.I., 0.6-7.6). These data suggest that the G allele behaves as a dominant allele with more than an additive effect of allele copy number (2.2+2.2−1<5.2).

For white men with prostate cancer, the odds ratio for the G allele as a risk factor for disease is 3.3 (95% CI, 0.6-18.3). The odds ratio for the homozygote (GG) is actually less than 1, whereas the heterozygote (GT genotype) has a remarkable odds ratio of 23.2 (95% C.I., 2.7-201). These data suggest that the G allele behaves as a codominant allele.

For black men with NIDDM, the odds ratio for the T allele as a risk factor for disease is 1.6 (95% CI, 0.4-5.8). The odds ratio for the homozygote (TT) is 1.5 (95% CI, 0.1-15.5), whereas the heterozygote (TG genotype) has an odds ratio of less than 1. These data suggest that the T allele behaves as a recessive allele.

For black women with NIDDM, the odds ratio for the T allele as a risk factor for disease is 2.3 (95% CI, 0.7-8.3). The odds ratio for the homozygote (TT) is 5.0 (95% CI, 0.4-64.4), whereas the heterozygote (TG genotype) has an odds ratio of 5.3 (95% CI, 0.4-75.8). These data suggest that the T allele behaves as a classical dominant allele.

For white men with NIDDM, the odds ratio for the G allele as a risk factor for disease is 3.0 (95% CI, 0.5-17.2). The odds ratio for the homozygote (GG) is 1.9 (95% CI, 0.4-9.3), whereas the heterozygote (GT genotype) has an odds ratio of 5.7 (95% CI, 0.6-54.1). These data suggest that the G allele behaves as a codominant allele.

For white women with NIDDM, the odds ratio for the G allele as a risk factor for disease is 3.8 (95% CI, 0.8-17.2). The odds ratio for the homozygote (GG) is 8.6 (95% CI, 0.9-83.8), whereas the heterozygote (GT genotype) has an odds ratio of only 1.7 (95% CI, 0.5-6.2). These data suggest that the G allele behaves as a dominant allele, with a more than multiplicative effect of allele dosage [8.6>>(1.7)(1.7)].

For white men with ESRD due to NIDDM, the odds ratio for the T allele as a risk factor for disease is 3.4 (95% CI, 0.6-19.2) as compared with white men with NIDDM but no renal disease. The odds ratio for the homozygote (TT) is 5.7 (95% CI, 0.6-54.1), while the heterozygote (TG genotype) has a similar odds ratio of 5.0 (95% CI, 0.4-59.7). These data suggest that the T allele behaves as a classical dominant allele.

For white women with ESRD due to NIDDM, the odds ratio for the T allele as a risk factor for disease is a remarkable 18.3 (95% CI, 2.3-148) as compared with white women with NIDDM but no renal disease. The odds ratio for the homozygote (TT) is 7.7 (95% CI, 0.8-75.3), while the heterozygote (TG genotype) has an odds ratio of only 0.7. These data suggest that the T allele behaves as a classical recessive allele.

For Caucasians with alcohol abuse the odds ratio for the G allele was 2.1 (95% CI, 0.9-5.2). Data were not sufficient to generate genotypic odds ratios of 1.5 or greater. These data further suggest that the EDN-1 gene is significantly associated with alcohol abuse in Caucasians, i.e. abnormal activity of the EDN-1 gene predisposes Caucasians to alcohol abuse.

For Caucasians with colon cancer the odds ratio for the G allele was 1.6 (95 % CI, 0.6-4.2). Data were not sufficient to generate genotypic odds ratios of 1.5 or greater. These data further suggest that the EDN-1 gene is significantly associated with colon cancer in Caucasians, i.e. abnormal activity of the EDN-1 gene predisposes Caucasians to colon cancer.

For Caucasians with diabetic cardiomyopathy the odds ratio for the T allele was 2.1 (95% CI, 0.7-6.2), compared to Caucasians with MI due to NIDDM. Data were not sufficient to generate genotypic odds ratios of 1.5 or greater. These data further suggest that the EDN-1 gene is significantly associated with diabetic cardiomyopathy in Caucasians, i.e. abnormal activity of the EDN-1 gene predisposes Caucasians to diabetic cardiomyopathy.

For Caucasians with ESRD due to hypertension the odds ratio for the G allele was 2.6 (95% CI, 0.8-9.1), compared to Caucasians with hypertension only. The odds ratio for the homozygote (G/G) was 0.8^{H} (95% CI, 0-14.1), while the odds ratio for the heterozygote (T/G) was 2.3^{H} (95% CI, 0-137). These data suggest that G allele acts in a co-dominant manner in this patient population. These data further suggest that the EDN-1 gene is significantly associated with ESRD due to hypertension in Caucasians, i.e. abnormal activity of the EDN-1 gene predisposes Caucasians to ESRD due to hypertension.

For Caucasians with ESRD due to FSGS the odds ratio for the G allele was 1.7 (95% CI, 0.7-4.5). The odds ratio for the homozygote (G/G) was 1.1^{H} (95% CI, 0.1-19.7), while the odds ratio for the heterozygote (T/G) was 2.7^{H} (95% CI, 0.1-75). These data suggest that the G allele acts in a co-dominant manner in this patient population. These data further suggest that the EDN-1 gene is significantly associated with ESRD due to FSGS in Caucasians, i.e. abnormal activity of the EDN-1 gene predisposes Caucasians to ESRD due to FSGS.

For Caucasians with hypertension only the odds ratio for the T allele was 1.6 (95% CI, 0.5-5.2). The odds ratio for the homozygote (TIT ) was 1.7^{H} (95% CI, 0.1-28.6), while the odds ratio for the heterozygote (T/G) was 1.3^{H} (95% CI, 0-38). These data suggest that the T allele acts in a recessive manner in this patient population. These data further suggest that the EDN-1 gene is significantly associated with hypertension only in Caucasians, i.e. abnormal activity of the EDN-1 gene predisposes Caucasians to hypertension only.

For Caucasians with CVA due to HTN the odds ratio for the G allele was 2.1 (95% CI, 0.6-7.6), compared to Caucasians with hypertension only. Data were not sufficient to generate genotypic odds ratios of 1.5 or greater. These data further suggest that the EDN-1 gene is significantly associated with CVA due to HTN in Caucasians, i.e. abnormal activity of the EDN-1 gene predisposes Caucasians to CVA due to HTN.

The binding site of T-cell factor-2 alpha (TCF-2 alpha) is predicted to be disrupted by the T2239→G SNP (Quandt K et al., Nucleic Acids Res., 23:4878-4884, 1995). TCF-2 alpha binds to a core sequence of five nucleotides, 5′-KTKTC-3′ (Waterman M. L., et al. New Biology, 2(7):621-636, 1990). A TCF-2 alpha binding site, which occurs on average 3.91 times per 1000 base pairs of random genomic sequence in vertebrates, is predicted to occur at position 2236 to 2240 on the (−) strand of reference sequence J05008.1 (matrix score 1.000, with 1.000 being an identical match). The T2239→G SNP replaces the indicated T with a G within the core binding sequence.

TCF-2 alpha is a transcriptional activator in lymphoid cells, although nothing is known of its activity in other cell types. Disruption of the TCF-2 alpha core binding site is expected to result in a decreased rate of transcription of the endothelin-1 gene.

TABLE 9
GROUP I ALLELE FREQUENCIES

| | |
|---|---|
| A | C |

CONTROL

| | | | | |
|---|---|---|---|---|
| Black men (n = 46 chromosomes) | 30 | (65%) | 16 | (35%) |
| Black women (n = 40 chromosomes) | 30 | (75%) | 10 | (25%) |
| White men (n = 42 chromosomes) | 38 | (90%) | 4 | (10%) |
| White women (n = 48 chromosomes) | 42 | (88%) | 6 | (13%) |

DISEASE
Breast Cancer

| | | | | |
|---|---|---|---|---|
| Black women (n = 16 chromosomes) | 7 | (44%) | 9 | (56%) |
| White women (n = 12 chromosomes) | 9 | (75%) | 3 | (25%) |

Lung Cancer

| | | | | |
|---|---|---|---|---|
| Black men (n = 20 chromosomes) | 13 | (65%) | 7 | (35%) |
| Black women (n = 16 chromosomes) | 11 | (69%) | 5 | (31%) |
| White men (n = 20 chromosomes) | 13 | (65%) | 7 | (35%) |
| White women (n = 12 chromosomes) | 8 | (67%) | 4 | (33%) |

Prostate Cancer

| | | | | |
|---|---|---|---|---|
| Black men (n = 16 chromosomes) | 13 | (81%) | 3 | (19%) |
| White men (n = 18 chromosomes) | 11 | (61%) | 7 | (39%) |

HTN

| | | | | |
|---|---|---|---|---|
| Black men (n = 18 chromosomes) | 12 | (67%) | 6 | (33%) |
| Black women (n = 16 chromosomes) | 13 | (81%) | 3 | (19%) |
| White men (n = 22 chromosomes) | 21 | (95%) | 1 | (5%) |
| White women (n = 18 chromosomes) | 15 | (83%) | 3 | (17%) |

ESRD due to HTN

| | | | | |
|---|---|---|---|---|
| Black men (n = 12 chromosomes) | 10 | (83%) | 2 | (17%) |
| Black women (n = 10 chromosomes) | 6 | (60%) | 4 | (40%) |
| White men (n = 14 chromosomes) | 12 | (86%) | 2 | (14%) |
| White women (n = 4 chromosomes) | 4 | (100%) | 0 | (0%) |

NIDDM

| | | | | |
|---|---|---|---|---|
| Black men (n = 16 chromosomes) | 13 | (81%) | 3 | (19%) |
| Black women (n = 16 chromosomes) | 11 | (69%) | 5 | (31%) |
| White men (n = 22 chromosomes) | 16 | (73%) | 6 | (27%) |
| White women (n = 20 chromosomes) | 15 | (75%) | 5 | (25%) |

ESRD due to NIDDM

| | | | | |
|---|---|---|---|---|
| Black men (n = 4 chromosomes) | 3 | (75%) | 1 | (25%) |
| Black women (n = 18 chromosomes) | 14 | (78%) | 4 | (22%) |
| White men (n = 16 chromosomes) | 14 | (88%) | 2 | (13%) |
| White women (n = 10 chromosomes) | 10 | (100%) | 0 | (0%) |

TABLE 10
GROUP II ALLELE FREQUENCIES

| Disease | Race | CHROMOSOMES | N | C | N | A |
|---|---|---|---|---|---|---|
| Controls | African-American | 90 | 25 | 27.8% | 65 | 72.2% |
| | Caucasian | 90 | 15 | 16.7% | 75 | 83.3% |
| Colon cancer | African-American | 48 | 8 | 16.7% | 40 | 83.3% |
| | Caucasian | 44 | 7 | 15.9% | 37 | 84.1% |
| Hypertension | African-American | 42 | 6 | 14.3% | 36 | 85.7% |
| | Caucasian | 44 | 4 | 9.1% | 40 | 90.9% |
| ASPVD due to HTN | African-American | 50 | 10 | 20.0% | 40 | 80.0% |
| | Caucasian | 50 | 7 | 14.0% | 43 | 86.0% |
| CVA due to HTN | Caucasian | 48 | 9 | 18.8% | 39 | 81.3% |
| Cataracts due to HTN | African-American | 44 | 9 | 20.5% | 35 | 79.5% |
| HTN CM | African-American | 1 | 7 | 14.6% | 41 | 85.4% |
| | Caucasian | 44 | 5 | 11.4% | 39 | 88.6% |
| MI due to HTN | African-American | 42 | 11 | 26.2% | 31 | 73.8% |
| | Caucasian | 46 | 11 | 23.9% | 35 | 76.1% |
| NIDDM | African-American | 44 | 11 | 25.0% | 33 | 75.0% |
| | Caucasian | 48 | 13 | 27.1% | 35 | 72.9% |
| ASPVD due to NIDDM | African-American | 46 | 15 | 32.6% | 31 | 67.4% |
| | Caucasian | 46 | 8 | 17.4% | 38 | 82.6% |
| CVA due to NIDDM | African-American | 48 | 9 | 18.8% | 39 | 81.3% |
| | Caucasian | 46 | 5 | 10.9% | 41 | 89.1% |
| Ischemic CM | African-American | 48 | 11 | 22.9% | 37 | 77.1% |
| | Caucasian | 42 | 8 | 19.0% | 34 | 81.0% |
| Ischemic CM with NIDDM | African-American | 48 | 14 | 29.2% | 34 | 70.8% |
| | Caucasian | 48 | 7 | 14.6% | 41 | 85.4% |
| MI due to NIDDM | African-American | 48 | 6 | 12.5% | 42 | 87.5% |
| | Caucasian | 46 | 10 | 21.7% | 36 | 78.3% |
| Afib without valvular disease | African-American | 48 | 14 | 29.2% | 34 | 70.8% |
| | Caucasian | 48 | 8 | 16.7% | 40 | 83.3% |
| Alcohol abuse | African-American | 48 | 17 | 35.4% | 31 | 64.6% |
| | Caucasian | 48 | 12 | 25.0% | 36 | 75.0% |
| Anxiety | African-American | 48 | 16 | 33.3% | 32 | 66.7% |
| | Caucasian | 42 | 10 | 23.8% | 32 | 76.2% |
| Asthma | African-American | 48 | 11 | 22.9% | 37 | 77.1% |
| | Caucasian | 48 | 6 | 12.5% | 42 | 87.5% |
| COPD | African-American | 44 | 3 | 6.8% | 41 | 93.2% |
| | Caucasian | 42 | 8 | 19.0% | 34 | 81.0% |
| Cholecystectomy | African-American | 48 | 14 | 29.2% | 34 | 70.8% |
| | Caucasian | 46 | 7 | 15.2% | 39 | 84.8% |
| DJD | African-American | 40 | 9 | 22.5% | 31 | 77.5% |
| ESRD and frequent de-clots | African-American | 46 | 13 | 28.3% | 33 | 71.7% |
| | Caucasian | 42 | 5 | 11.9% | 37 | 88.1% |
| ESRD due to FSGS | African-American | 44 | 13 | 29.5% | 31 | 70.5% |
| | Caucasian | 46 | 10 | 21.7% | 36 | 78.3% |
| ESRD due to IDDM | African-American | 48 | 14 | 29.2% | 34 | 70.8% |
| | Caucasian | 44 | 3 | 6.8% | 41 | 93.2% |
| Seizure disorder | African-American | 46 | 19 | 41.3% | 27 | 58.7% |
| | Caucasian | 48 | 5 | 10.4% | 43 | 89.6% |

TABLE 11
GROUP I GENOTYPE FREQUENCIES

| | | |
|---|---|---|
| A/A | A/C | C/C |

CONTROLS

| | | | | | | |
|---|---|---|---|---|---|---|
| Black men (n = 23) | 10 | (43%) | 10 | (43%) | 3 | (13%) |
| Black women (n = 20) | 11 | (55%) | 8 | (40%) | 1 | (5%) |
| White men (n = 21) | 18 | (86%) | 2 | (10%) | 1 | (5%) |
| White women (n = 24) | 19 | (79%) | 4 | (17%) | 1 | (4%) |

DISEASE
Breast Cancer

| | | | | | | |
|---|---|---|---|---|---|---|
| Black women (n = 8) | 3 | (38%) | 1 | (13%) | 4 | (50%) |
| White women (n = 6) | 4 | (67%) | 1 | (17%) | 1 | (17%) |

Lung Cancer

| | | | | | | |
|---|---|---|---|---|---|---|
| Black men (n = 10) | 5 | (50%) | 3 | (30%) | 2 | (20%) |
| Black women (n = 8) | 4 | (50%) | 3 | (38%) | 1 | (13%) |
| White men (n = 10) | 5 | (50%) | 3 | (30%) | 2 | (20%) |
| White women (n = 6) | 2 | (33%) | 4 | (67%) | 0 | (0%) |

Prostate Cancer

| | | | | | | |
|---|---|---|---|---|---|---|
| Black men (n = 8) | 5 | (63%) | 3 | (38%) | 0 | (0%) |
| White men (n = 9) | 3 | (33%) | 5 | (56%) | 1 | (11%) |

HTN

| | | | | | | |
|---|---|---|---|---|---|---|
| Black men (n = 9) | 5 | (56%) | 2 | (22%) | 2 | (22%) |
| Black women (n = 8) | 5 | (63%) | 3 | (38%) | 0 | (0%) |
| White men (n = 11) | 10 | (91%) | 1 | (9%) | 0 | (0%) |
| White women (n = 9) | 7 | (78%) | 1 | (11%) | 1 | (11%) |

ESRD due to HTN

| | | | | | | |
|---|---|---|---|---|---|---|
| Black men (n = 6) | 4 | (67%) | 2 | (33%) | 0 | (0%) |
| Black women (n = 5) | 3 | (60%) | 0 | (0%) | 2 | (40%) |
| White men (n = 7) | 5 | (71%) | 2 | (29%) | 0 | (0%) |
| White women (n = 2) | 2 | (100%) | 0 | (0%) | 0 | (0%) |

NIDDM

| | | | | | | |
|---|---|---|---|---|---|---|
| Black men (n = 8) | 5 | (63%) | 3 | (38%) | 0 | (0%) |
| Black women (n = 8) | 4 | (50%) | 3 | (38%) | 1 | (13%) |
| White men (n = 11) | 7 | (64%) | 2 | (18%) | 2 | (18%) |
| White women (n = 10) | 6 | (60%) | 3 | (30%) | 1 | (10%) |

ESRD due to NIDDM

| | | | | | | |
|---|---|---|---|---|---|---|
| Black men (n = 2) | 1 | (50%) | 1 | (50%) | 0 | (0%) |
| Black women (n = 9) | 5 | (56%) | 4 | (44%) | 0 | (0%) |
| White men (n = 8) | 6 | (75%) | 2 | (25%) | 0 | (0%) |
| White women (n = 5) | 5 | (100%) | 0 | (0%) | 0 | (0%) |

TABLE 12
GROUP II GENOTYPE FREQUENCIES

| Disease | Race | People | N | C/C | N | C/A | N | A/A |
|---|---|---|---|---|---|---|---|---|
| Controls | African-American | 45 | 4 | 8.9% | 17 | 37.8% | 24 | 53.3% |
| | Caucasian | 45 | 3 | 6.7% | 9 | 20.0% | 33 | 73.3% |
| Colon cancer | African-American | 24 | 0 | 0.0% | 8 | 33.3% | 16 | 66.7% |
| | Caucasian | 22 | 1 | 4.5% | 5 | 22.7% | 16 | 72.7% |
| Hypertension | African-American | 21 | 0 | 0.0% | 6 | 28.6% | 15 | 71.4% |
| | Caucasian | 22 | 0 | 0.0% | 4 | 18.2% | 18 | 81.8% |
| ASPVD due to HTN | African-American | 25 | 2 | 8.0% | 6 | 24.0% | 17 | 68.0% |
| | Caucasian | 25 | 1 | 4.0% | 5 | 20.0% | 19 | 76.0% |
| CVA due to HTN | Caucasian | 24 | 1 | 4.2% | 7 | 29.2% | 16 | 66.7% |
| Cataracts due to HTN | African-American | 22 | 1 | 4.5% | 7 | 31.8% | 14 | 63.6% |
| ESRD due to HTN | African-American | 22 | 4 | 18.2% | 8 | 36.4% | 10 | 45.5% |
| | Caucasian | 24 | 1 | 4.2% | 10 | 41.7% | 13 | 54.2% |
| HTN CM | African-American | 24 | 2 | 8.3% | 3 | 12.5% | 19 | 79.2% |
| | Caucasian | 22 | 0 | 0.0% | 5 | 22.7% | 17 | 77.3% |
| MI due to HTN | African-American | 21 | 2 | 9.5% | 7 | 33.3% | 12 | 57.1% |
| | Caucasian | 23 | 2 | 8.7% | 7 | 30.4% | 14 | 60.9% |
| NIDDM | African-American | 22 | 2 | 9.1% | 7 | 31.8% | 13 | 59.1% |
| | Caucasian | 24 | 5 | 20.8% | 3 | 12.5% | 16 | 66.7% |
| ASPVD due to NIDDM | African-American | 23 | 2 | 8.7% | 11 | 47.8% | 10 | 43.5% |
| | Caucasian | 23 | 1 | 4.3% | 6 | 26.1% | 16 | 69.6% |
| CVA due to NIDDM | African-American | 24 | 0 | 0.0% | 9 | 37.5% | 15 | 62.5% |
| | Caucasian | 23 | 0 | 0.0% | 5 | 21.7% | 18 | 78.3% |
| Ischemic CM | African-American | 24 | 2 | 8.3% | 7 | 29.2% | 15 | 62.5% |
| | Caucasian | 21 | 1 | 4.8% | 6 | 28.6% | 14 | 66.7% |
| Ischemic CM with NIDDM | African-American | 24 | 3 | 12.5% | 8 | 33.3% | 13 | 54.2% |
| | Caucasian | 24 | 0 | 0.0% | 7 | 29.2% | 17 | 70.8% |
| MI due to NIDDM | African-American | 24 | 0 | 0.0% | 6 | 25.0% | 18 | 75.0% |
| | Caucasian | 23 | 0 | 0.0% | 10 | 43.5% | 13 | 56.5% |
| Afib without valvular disease | African-American | 24 | 1 | 4.2% | 12 | 50.0% | 11 | 45.8% |
| | Caucasian | 24 | 0 | 0.0% | 8 | 33.3% | 16 | 66.7% |
| Alcohol abuse | African-American | 24 | 5 | 20.8% | 7 | 29.2% | 12 | 50.0% |
| | Caucasian | 24 | 2 | 8.3% | 8 | 33.3% | 14 | 58.3% |
| Anxiety | African-American | 24 | 3 | 12.5% | 10 | 41.7% | 11 | 45.8% |
| | Caucasian | 21 | 0 | 0.0% | 10 | 47.6% | 11 | 52.4% |
| Asthma | African-American | 24 | 2 | 8.3% | 7 | 29.2% | 15 | 62.5% |
| | Caucasian | 24 | 0 | 0.0% | 6 | 25.0% | 18 | 75.0% |
| COPD | African-American | 22 | 0 | 0.0% | 3 | 13.6% | 19 | 86.4% |
| | Caucasian | 21 | 1 | 4.8% | 6 | 28.6% | 14 | 66.7% |
| Cholecystectomy | African-American | 24 | 1 | 4.2% | 12 | 50.0% | 11 | 45.8% |
| | Caucasian | 23 | 0 | 0.0% | 7 | 30.4% | 16 | 69.6% |
| DJD | African-American | 20 | 1 | 5.0% | 7 | 35.0% | 12 | 60.0% |
| ESRD and frequent de-clots | African-American | 23 | 3 | 13.0% | 7 | 30.4% | 13 | 56.5% |
| | Caucasian | 21 | 1 | 4.8% | 3 | 14.3% | 17 | 81.0% |
| ESRD due to FSGS | African-American | 22 | 1 | 4.5% | 11 | 50.0% | 10 | 45.5% |
| | Caucasian | 23 | 0 | 0.0% | 10 | 43.5% | 13 | 56.5% |
| ESRD due to IDDM | African-American | 24 | 3 | 12.5% | 8 | 33.3% | 13 | 54.2% |
| | Caucasian | 22 | 0 | 0.0% | 3 | 13.6% | 19 | 86.4% |
| Seizure disorder | African-American | 23 | 4 | 17.4% | 11 | 47.8% | 8 | 34.8% |
| | Caucasian | 24 | 0 | 0.0% | 5 | 20.8% | 19 | 79.2% |

Allele-Specific Odds Ratios

The susceptibility allele is indicated below, as well as the odds ratio (OR). Where there was a “0” in a cell which produced a 0 in the denominator, Haldane's correction (multiplying all cells by 2 and adding 1) was used. If the odds ratio (OR) was ≧1.5, the 95% confidence interval (C.I.) is also given. An odds ratio of 1.5 was chosen as the threshold of significance based on the recommendation of Austin et al. in Epideyniol. Rev., 16:65-76, 1994. “[E]pidemiology in general and case-control studies in particular are not well suited for detecting weak associations (odds ratios <1.5).” Id. at 66. Odds ratios of greater than 1.5 are highlighted below.

TABLE 13
GROUP I ALLELE-SPECIFIC ODDS RATIOS

| | SUSCEPTIBILITY | | |
|---|---|---|---|
| DISEASE | ALLELE | OR | 95% C.I. |
| Breast Cancer | | | |
| Black women | C | 3.9 | 1.1-13 |
| White women | C | 2.3 | 0.5-11 |
| Lung Cancer | | | |
| Black men | C | 1.0 | 0.3-3.0 |
| Black women | C | 1.4 | 0.4-4.9 |
| White men | C | 5.1 | 1.3-20 |
| White women | C | 3.5 | 0.8-15 |
| Prostate Cancer | | | |
| Black men | A | 2.3 | 0.6-9.3 |
| White men | C | 6.0 | 1.5-25 |
| Hypertension (HTN) | | | |
| Black men | A | 1.1 | 0.3-3.4 |
| Black women | A | 1.4 | 0.3-6.1 |
| White men | A | 2.2 | 0.2-21 |
| White women | C | 1.4 | 0.3-6.3 |
| ESRD due to HTN* | | | |
| Black men | A | 2.5 | 0.4-15.2 |
| Black women | C | 2.9 | 0.5-17.2 |
| White men | C | 3.5 | 0.3-42.8 |
| White women | A | 2.0^{H} | 0.2-18.8 |
| NIDDM | | | |
| Black men | A | 2.3 | 0.6-9.3 |
| Black women | C | 1.4 | |
| White men | C | 3.6 | 0.9-14.4 |
| White women | C | 2.3 | 0.6-8.8 |
| ESRD due to NIDDM*¹ | | | |
| Black men | C | 1.4 | |
| Black women | A | 1.6 | 0.3-7.4 |
| White men | A | 2.6 | 0.5-15.2 |
| White women | A | 7.5^{H} | 0.9-62.1 |

*Compared to HTN alone.
*¹Compared to NIDDM alone.

TABLE 14
GROUP II ALLELE-SPECIFIC ODDS RATIOS

| | | | | Lower | Upper | |
|---|---|---|---|---|---|---|
| | | Risk | Odds | Limit | Limit | |
| Disease | Race | Allele | Ratio | 95% CI | 95% CI | Haldane |
| Colon cancer | African-American | A | 1.9 | 0.8 | 4.7 | |
| | Caucasian | A | 1.1 | 0.4 | 2.8 | |
| ASPVD due to HTN* | African-American | C | 1.5 | 0.5 | 4.5 | |
| | Caucasian | C | 1.6 | 0.4 | 6.0 | |
| CVA due to HTN* | Caucasian | C | 2.3 | 0.7 | 8.1 | |
| Cataracts due to HTN* | African-American | A | 1.5 | 0.6 | 3.6 | |
| HTN CM*¹ | African-American | A | 2.1 | 0.7 | 6.0 | |
| | Caucasian | A | 2.5 | 0.8 | 7.8 | |
| MI due to HTN* | African-American | C | 2.1 | 0.7 | 6.4 | |
| | Caucasian | C | 3.1 | 0.9 | 10.8 | |
| ASPVD due to NIDDM*² | African-American | C | 1.5 | 0.6 | 3.6 | |
| | Caucasian | A | 1.8 | 0.7 | 4.8 | |
| CVA due to NIDDM*² | African-American | A | 1.4 | 0.5 | 3.9 | |
| | Caucasian | A | 3.0 | 1.0 | 9.4 | |
| Ischemic CM with NIDDM*³ | African-American | C | 2.9 | 1.0 | 8.3 | |
| | Caucasian | A | 1.6 | 0.6 | 4.7 | |
| MI due to NIDDM*² | African-American | A | 2.3 | 0.8 | 7.0 | |
| | Caucasian | A | 1.3 | 0.5 | 3.4 | |
| Afib without | African-American | C | 1.1 | 0.5 | 2.3 | |
| valvular disease | Caucasian | A | 1.0 | 0.4 | 2.6 | |
| Alcohol abuse | African-American | C | 1.4 | 0.7 | 3.0 | |
| | Caucasian | C | 1.7 | 0.7 | 3.9 | |
| Anxiety | African-American | C | 1.3 | 0.6 | 2.8 | |
| | Caucasian | C | 1.6 | 0.6 | 3.8 | |
| Asthma | African-American | A | 1.3 | 0.6 | 2.9 | |
| | Caucasian | A | 1.4 | 0.5 | 3.9 | |
| COPD | African-American | A | 5.3 | 1.5 | 18.5 | |
| | Caucasian | C | 1.2 | 0.5 | 3.0 | |
| Cholecystectomy | African-American | C | 1.1 | 0.5 | 2.3 | |
| | Caucasian | A | 1.1 | 0.4 | 3.0 | |
| DJD | African-American | A | 1.3 | 0.6 | 3.2 | |
| ESRD and frequent | African-American | C | 1.0 | 0.5 | 2.3 | |
| de-clots | Caucasian | A | 1.5 | 0.5 | 4.4 | |
| ESRD due to FSGS | African-American | C | 1.1 | 0.5 | 2.4 | |
| | Caucasian | C | 1.4 | 0.6 | 3.4 | |
| ESRD due to IDDM | African-American | C | 1.1 | 0.5 | 2.3 | |
| | Caucasian | A | 2.7 | 0.7 | 10.0 | |
| Seizure disorder | African-American | C | 1.8 | 0.9 | 3.9 | |
| | Caucasian | A | 1.7 | 0.6 | 5.1 | |

*Compared to HTN alone.
*¹Compared to MI with HTN.
*²Compared to NIDDM alone.
*³Compared to MI with NIDDM.

Genotype-Specific Odds Ratios

The susceptibility allele (S) is indicated; the alternative allele at this locus is defined as the protective allele (P). Also presented is the odds ratio (OR) for each genotype (SS, SP). The odds ratio for the PP genotype is 1, since it is the reference group, and is not presented separately. For odds ratios ≧1.5, the 95% confidence interval (C.I.) is also given, in parentheses. An odds ratio of 1.5 was chosen as the threshold of significance based on the recommendation of Austin et al., in Epideniiol. Rev., 16:65-76, 1994. “[Epidemiology in general and case-control studies in particular are not well suited for detecting weak associations (odds ratios <1.5).” Id. at 66.

Haldane's zero cell correction was used when the denominator contained a zero. Odds ratios of greater than 1.5 are highlighted below.

TABLE 15
GROUP I GENOTYPE-SPECIFIC ODDS RATIOS

| | | | |
|---|---|---|---|
| | SUSCEPTI- | | |
| | BILITY | | |
| DISEASE | ALLELE | OR(SS) | OR(SP) |

Breast Cancer

| | | | | | |
|---|---|---|---|---|---|
| Black women | C | 14.7 | (1.2-185) | 0.5 | (0-5.3) |
| White women | C | 4.8 | (0.2-93) | 1.2 | (0.1-14) |

Lung Cancer

| | | | | | |
|---|---|---|---|---|---|
| White men | C | 7.2 | (0.5-97) | 5.4 | (0.7-42) |
| White women | C | 0 | | 9.5 | (1.3-71.0) |

Prostate Cancer

| | | | | | |
|---|---|---|---|---|---|
| Black men | A | 3.7 | (0.4-34)^{H} | 2.3 | (0.2-22)^{H} |
| White men | C | 6.0 | (0.3-124) | 15.0 | (1.9-116) |

Hypertension

| | | | | | |
|---|---|---|---|---|---|
| White men | A | 1.7 | (0.2-17)^{H} | 1.8 | (0.1-26)^{H} |

ESRD due
to HTN*

| | | | | | |
|---|---|---|---|---|---|
| Black men | A | 4.1 | (0.4-42)^{H} | 5.0 | (0.4-60)^{H} |
| Black women | C | 7.9 | (0.8-82)^{H} | 0 | |
| White men | C | 1.9 | (0.1-34)^{H} | 4.0 | (0.3-55.5)^{H} |
| White women | A | 1.0^{H} | | 1.0^{H} | |

NIDDM

| | | | | | |
|---|---|---|---|---|---|
| Black men | A | 3. 7 | (0.4-34)^{H} | 2.3 | (0.2-22)^{H} |
| White men | C | 5.1 | (0.4-66) | 2.6 | (0.3-22) |
| White women | C | 3.2 | (0.2-59) | 2.4 | (0.4-14) |

ESRD due
to NIDDM*¹

| | | | | | |
|---|---|---|---|---|---|
| Black women | A | 3.7 | (0.3-42)^{H} | 3.9 | (0.3-46) ^{H} |
| White men | A | 4.3 | (0.4-42)^{H} | 5.0 | (0.4-60)^{H} |
| White women | A | 2.5 | (0.2-28)^{H} | 0.4 | (0-9.4)^{H} |

TABLE 16
GROUP II GENOTYPE-SPECIFIC ODDS RATIOS

| | | RISK | SS | | SP | |
|---|---|---|---|---|---|---|
| Disease | Race | ALLELE | O.R. | HALDANE | O.R. | HALDANE |
| Colon cancer | African-American | A | 0.0 | | 0.7 | |
| | Caucasian | A | 0.7 | | 1.1 | |
| ASPVD due to HTN* | African-American | C | 4.4 | H | 0.9 | |
| | Caucasian | C | 2.8 | H | 1.2 | |
| CVA due to HTN* | Caucasian | C | 3.4 | H | 2.0 | |
| Cataracts due to HTN* | African-American | A | 0.4 | | 0.7 | |
| HTN CM*¹ | African-American | A | 0.6 | | 0.3 | |
| | Caucasian | A | 0.0 | | 0.6 | |
| MI due to HTN* | African-American | C | 6.2 | H | 1.5 | |
| | Caucasian | C | 6.4 | H | 2.3 | |
| ASPVD due to | African-American | C | 1.3 | | 2.0 | |
| NIDDM*² | Caucasian | A | 0.2 | | 2.0 | |
| CVA due to NIDDM*² | African-American | A | 0.0 | | 1.1 | |
| | Caucasian | A | 0.0 | | 1.5 | |
| Ischemic CM with | African-American | C | 9.6 | H | 1.8 | |
| NIDDM*³ | Caucasian | A | 0.8 | H | 0.5 | |
| MI due to NIDDM*² | African-American | A | 0.0 | | 0.6 | |
| | Caucasian | A | 0.0 | | 4.1 | |
| Afib without valvular | African-American | C | 0.5 | | 1.5 | |
| disease | Caucasian | A | 0.0 | | 1.8 | |
| Alcohol abuse | African-American | C | 2.5 | | 0.8 | |
| | Caucasian | C | 1.6 | | 2.1 | |
| Anxiety | African-American | C | 1.6 | | 1.3 | |
| | Caucasian | C | 0.0 | | 3.3 | |
| Asthma | African-American | A | 0.8 | | 0.7 | |
| | Caucasian | A | 0.0 | | 1.2 | |
| COPD | African-American | A | 0.0 | | 0.2 | |
| | Caucasian | C | 0.8 | | 1.6 | |
| Cholecystectomy | African-American | C | 0.5 | | 1.5 | |
| | Caucasian | A | 0.0 | | 1.6 | |
| DJD | African-American | A | 0.5 | | 0.8 | |
| ESRD and frequent | African-American | C | 1.4 | | 0.8 | |
| de-clots | Caucasian | A | 0.6 | | 0.6 | |
| ESRD due to FSGS | African-American | C | 0.6 | | 1.6 | |
| | Caucasian | C | 0.0 | | 2.8 | |
| ESRD due to IDDM | African-American | C | 1.4 | | 0.9 | |
| | Caucasian | A | 0.0 | | 0.6 | |
| Seizure disorder | African-American | C | 3.0 | | 1.9 | |
| | Caucasian | A | 0.0 | | 1.0 | |

*Compared to HTN alone.
*¹Compared to MI with HTN.
*²Compared to NIDDM alone.
*³Compared to MI with NIDDM.

PCR and sequencing were conducted as described in Example 1. The primers used were those in Example 1. The control samples were in good agreement with Hardy-Weinberg equilibrium, as follows:

For the Group I diseases, a frequency of 0.65 for the A allele (“p”) and 0.35 for the C allele (“q”) among black male control individuals predicts genotype frequencies of 42% A/A, 46% A/C, and 12% C/C at Hardy-Weinberg equilibrium (p2+2pq+q2=1). The observed genotype frequencies were 43% A/A, 43% A/C, and 13% C/C, in close agreement with those predicted for Hardy-Weinberg equilibrium.

A frequency of 0.75 for the A allele (“p”) and 0.25 for the C allele (“q”) among black female control individuals predicts genotype frequencies of 56% A/A, 38% A/C, and 6% C/C at Hardy-Weinberg equilibrium (p2+2pq+q2=1). The observed genotype frequencies were 55% A/A, 40% A/C, and 5% C/C, in very close agreement with those predicted for Hardy-Weinberg equilibrium.

A frequency of 0.90 for the A allele (“p”) and 0.10 for the C allele (“q”) among white male control individuals predicts genotype frequencies of 81% A/A, 18% A/C, and 1% C/C at Hardy-Weinberg equilibrium (p2+2pq+q2=1). The observed genotype frequencies were 86% A/A, 10% A/C, and 5% C/C, in reasonably close agreement with those predicted for Hardy-Weinberg equilibrium.

A frequency of 0.88 for the A allele (“p”) and 0.13 for the C allele (“q”) among white female control individuals predicts genotype frequencies of 77% A/A, 21% A/C, and 2% C/C at Hardy-Weinberg equilibrium (p2+2pq+q2=1). The observed genotype frequencies were 79% A/A, 17% A/C, and 4% C/C, in reasonably close agreement with those predicted for Hardy-Weinberg equilibrium.

For the Group II diseases, a frequency of 0.28 for the C allele (“p”) and 0.72 for the A allele (“q”) among African-American control individuals predicts genotype frequencies of 8.0% C/C, 40.0% C/A, and 52.0% A/A at Hardy-Weinberg equilibrium (p²+2pq+q²=1). The observed genotype frequencies were 8.9% C/C, 37.8% C/A, and 53.3% A/A, in excellent agreement with those predicted for Hardy-Weinberg equilibrium.

A frequency of 0.17 for the C allele (“i”) and 0.83 for the A allele (“q”) among Caucasian control individuals predicts genotype frequencies of 3.0% C/C, 28.0% C/A, and 69.0% A/A at Hardy-Weinberg equilibrium (p²+2pq+q²=1). The observed genotype frequencies were 6.7% C/C, 20.0% C/A, and 73.3% A/A, in excellent agreement with those predicted for Hardy-Weinberg equilibrium.

Using an allele-specific odds ratio of 1.5 or greater as a practical level of significance (see Austin H et al., discussed above), the following observations can be made.

For breast cancer among black women, the odds ratio for the C allele as a risk factor was 3.9 (95% CI, 1.1-13). The odds ratio for the homozygote (CC) was a remarkable 14.7 (95% CI, 1.2-185). The heterozygote (CA genotype) had an odds ratio indistinguishable from 1 (odds ratio 0.5; 95% C.I. 0-5.3), suggesting that the C allele behaves as a recessive allele in this patient population.

For breast cancer among white women the odds ratio for the C allele as a risk factor was 2.3 (95% CI, 0.5-11). The odds ratio for the homozygote (CC genotype) was 4.8 (95% CI, 0.2-93). The heterozygote (CA genotype) had an odds ratio indistinguishable from 1 (odds ratio 1.2; 95% C.I. 0.1-14), suggesting that the C allele behaves as a recessive allele in this patient population.

For lung cancer in white men the odds ratio for the C allele as a risk factor was 5.1 (95% CI, 1.3-20). The C allele displayed a dosage effect, with the heterozygote (AC) having an odds ratio of 5.4 (95% CI, 0.7-42), and the homozygote (CC) an odds ratio of 7.2 (95% CI, 0.5-97). These data are consistent with a dominant action of the C allele, since one copy is sufficient to increase the odds ratio from 1 (for the AA homozygote) to 5.4 (for the AC heterozygote). The increase to 7.2 represents less than an additive effect of the allele, since 5.4+5.4−1=9.8>7.2. These data are consistent with the C allele behaving as a dominant allele with interaction on less than an additive model.

For lung cancer in white women the odds ratio for the C allele (the novel SNP) as a risk factor was 3.5 (95% CI, 0.8-15). The CC homozygote surprisingly had a lower odds ratio, 0, than the heterozygote [9.5; 95% C.I., 1.3-71.0], suggesting that the C allele behaves other than as a simple recessive or dominant allele. The C allele may be codominant with the A allele.

For prostate cancer among black men the odds ratio for the reference A allele as a risk factor was 2.3 (95% CI, 0.6-9.3). The odds ratio for the homozygote (AA genotype) was 3.7^{H} (95% CI, 0.4-34). The heterozygote (AC genotype) had an odds ratio of 2.3^{H} (95% CI, 0.2-22). The A allele therefore behaves as a dominant allele, with an additive effect of increased allele dosage. The effect of the A allele on disease is as expected for an additive model (3.7˜2.3+2.3−1).

For prostate cancer in white men the odds ratio for the C allele (the novel SNP) as a risk factor was 6.0 (95% CI, 1.5-25). The CC homozygote surprisingly had a lower odds ratio (6.0; 95% CI, 0.3-124) than the heterozygote (15.0; 95% C.I. 1.9-116), suggesting that the C allele behaves other than as a simple dominant or recessive r lele. The C allele may be codominant with the A allele.

For hypertension among white men the odds ratio for the reference A allele as a risk factor was 2.2 (95% CI, 0.2-21). The odds ratio for the homozygote (AA genotype) was virtually the same (1.7^{H}; 95% CI,, 0.2-17) as that for the AC heterozygote (1.8^{H}; 95% CI, 0.1-26). These data indicate that the A allele behaves as a simple dominant allele, with no additional effect of a second copy of the allele.

For ESRD due to hypertension among black men the odds ratio for the reference A allele as a risk factor was 2.5 (95% CI, 0.4-15.2). The odds ratio for the homozygote (AA genotype) was 4.1^{H} (95% CI, 0.4-42), and that for the AC heterozygote was essentially the same [5.0^{H} (95% CI, 0.4-60)). These data suggest that the A allele behaves as a dominant allele.

For ESRD due to hypertension among black women the odds ratio for the C allele as a risk factor was 2.9 (95% CI, 0.5-17.2). The heterozygote (AC) had an odds ratio of 0, whereas the CC homozygote displayed an odds ratio of 7.9^{H} (95% CI, 0.8-82). These data are consistent with a recessive action of the C allele.

For ESRD due to hypertension among white men the odds ratio for the A allele as a risk factor was 3.5 (95% CI, 0.3-42.8). The odds ratio for the AC heterozygous genotype was 4.0 (95% CI, 0.3-55.5), and for the AA homozygous genotype was 1.9^{H} (95% CI, 0.1-34). The A allele appears to be codominant with the C allele.

For ESRD due to hypertension among white women the odds ratio for the A allele was 2.0^{H} (95% CI, 0.2-18.8), relative to white women with hypertension but no renal disease. The odds ratios for both the AC heterozygote and the AA homozygote were only 1.0 after the Haldane's correction, shedding no light on the mechanism of action of the A allele.

For NIDDM among black men, the odds ratio for the A allele at this locus was 2.3 (95% CI, 0.6-9.3). The odds ratio for the heterozygote was 2.3^{H} (95% CI, 0.2-22), and for the AA homozygote was 3.7^{H} (95% CI, 0.4-34). These data suggest that the A allele behaves as a dominant allele, with an additive effect of allele dosage (2.3+2.3−1˜3.7).

For NIDDM among white men, the odds ratio for the C allele at this locus was 3.6 (95% CI, 0.9-14.4). The odds ratio for the heterozygote was 2.6 (95% CI, 0.3-22), and for the CC homozygote was 5.1 (95% CI, 0.4-66). These data suggest that the C allele behaves as a dominant allele, with more than an additive effect of allele dosage (2.6+2.6−1<5.1).

For NIDDM among white women, the odds ratio for the C allele at this locus was 2.3 (95% CI, 0.6-8.8). The odds ratio for the heterozygote was 2.4 (95% CI, 0.4-14), and for the CC homozygote was 3.2 (95% CI, 0.2-59). These data suggest that the C allele behaves as a dominant allele, with approximately an additive effect of allele dosage (2.4+2.4−1=3.8˜3.2).

For ESRD due to NIDDM among black women, the odds ratio for the A allele at this locus was 1.6 (95% CI, 0.3-7.4). The odds ratio for the heterozygote was 3.9^{H} (95% CI, 0.3-46), and for the AA homozygote was 3.7^{H} (95% CI, 0.3-42). These data suggest that the A allele behaves as a classic dominant allele, with no effect of allele dosage.

For ESRD due to NIDDM among white men, the odds ratio for the A allele at this locus was 2.6 (95% CI, 0.5-15.2). The odds ratio for the heterozygote was 5.0^{H} (95% CI, 0.4-60), and for the AA homozygote was 4.3^{H} (95% CI, 0.4-42). These data suggest that the A allele behaves as a classic dominant allele, with no effect of allele dosage.

For ESRD due to NIDDM among white women, the odds ratio for the A allele at this locus was 7.5 (95% CI, 0.9-62.1). The odds ratio for the heterozygote was indistinguishable from 1, and for the AA homozygote was 2.5^{H} (95% CI, 0.2-28). These data suggest that the A allele behaves as a recessive allele.

For Caucasians with a history of alcohol abuse the odds ratio for the C allele was 1.7 (95% CI, 0.7-3.9). The odds ratio for the homozygote (C/C) was 1.6 (95% CI, 0.2-10.5), while the odds ratio for the heterozygote (C/ A) was 2.1 (95% CI, 0.7-6.5). These data suggest that the C allele acts in a co-dominant manner in this patient population. These data further suggest that the EDN-1 gene is significantly associated with alcohol abuse in Caucasians, i.e. abnormal activity of the EDN-1 gene predisposes Caucasians to alcohol abuse.

For Caucasians with anxiety the odds ratio for the C allele was 1.6 (95% CI, 0.6-3.8). The odds ratio for the homozygote (C/C) was 0, while the odds ratio for the heterozygote (C/A) was 3.3 (95% CI, 1.1-10.3). These data suggest that the C allele acts in a co-dominant manner in this patient population. These data further suggest that the EDN-1 gene is significantly associated with anxiety in Caucasians, i.e. abnormal activity of the EDN-1 gene predisposes Caucasians to anxiety.

For Caucasians with ASPVD due to NIDDM the odds ratio for the A allele was 1.8 (95% CI, 0.7-4.8), compared to Caucasians with NIDDM only. The odds ratio for the homozygote (A/A) was 0.2 (95% CI, 0-1.9), while the odds ratio for the heterozygote (C/A) was 2.0 (95% CI, 0.4-9.4). These data suggest that the A allele acts in a co-dominant manner in this patient population. These data further suggest that the EDN-1 gene is significantly associated with ASPVD due to NIDDM in Caucasians, i.e. abnormal activity of the EDN-1 gene predisposes Caucasians to ASPVD due to NIDDM.

For African-Americans with colon cancer the odds ratio for the A allele was 1.9 (95% CI, 0.8-4.7). Data were not sufficient to generate genotypic odds ratios of 1.5 or greater. These data further suggest that the EDN-1 gene is significantly associated with colon cancer in African-Americans, i.e. abnormal activity of the EDN-1 gene predisposes African-Americans to colon cancer.

For African-Americans with COPD the odds ratio for the A allele was 5.3 (95% CI, 1.5-18.5). Data were not sufficient to generate genotypic odds ratios of 1.5 or greater. These data further suggest that the EDN-1 gene is significantly associated with COPD in African-Americans, i.e. abnormal activity of the EDN-1 gene predisposes African-Americans to COPD.

For African-Americans with diabetic cardiomyopathy the odds ratio for the C allele was 2.9 (95% CI, 1-8.3), compared to African-Americans with MI due to NIDDM. The odds ratio for the homozygote (C/C) was 9.6^{H} (95% CI, 0.2-574.5), while the odds ratio for the heterozygote (C/A) was 1.8 (95% CI, 0.5-6.6).These data suggest that the C allele acts in a dominant manner in this patient population. These data further suggest that the EDN-1 gene is significantly associated with diabetic cardiomyopathy in African-Americans, i.e. abnormal activity of the EDN-1 gene predisposes African-Americans to diabetic cardiomyopathy.

For Caucasians with diabetic cardiomyopathy the odds ratio for the A allele was 1.6 (95% CI, 0.6-4.7), compared to Caucasians with MI due to NIDDM. Data were not sufficient to generate genotypic odds ratios of 1.5 or greater. These data further suggest that the EDN-1 gene is significantly associated with diabetic cardiomyopathy in Caucasians, i.e. abnormal activity of the EDN-1 gene predisposes Caucasians to diabetic cardiomyopathy.

For Caucasians with ESRD due to IDDM the odds ratio for the A allele was 2.7 (95% CI, 0.7-10). Data were not sufficient to generate genotypic odds ratios of 1.5 or greater. These data further suggest that the EDN-1 gene is significantly associated with ESRD due to IDDM in Caucasians, i.e. abnormal activity of the EDN-1 gene predisposes Caucasians to ESRD due to IDDM.

For African-Americans with ESRD due to IDDM the odds ratio for the C allele was 1.6 (95% CI, 0.6-3.9), compared to African-Americans with NIDDM only. The odds ratio for the homozygote (C/C) was 2.0 (95% CI, 0.3-14), while the odds ratio for the heterozygote (C/A) was 1.7 (95% CI, 0.5-6.1). These data suggest that the C allele acts in a dominant manner in this patient population with a less than additive effect of allele dosage [2<3.4=(1.7+1.7−1.0)]. (Goldstein et al., Monogr. Natl. Cancer Inst.; 26:49-54, 1999). These data further suggest that the EDN-1 gene is significantly associated with ESRD due to IDDM in African-Americans, i.e. abnormal activity of the EDN-1 gene predisposes African-Americans to ESRD due to IDDM.

For African-Americans with hypertensive cardiomyopathy the odds ratio for the A allele was 2.1 (95% CI, 0.7-6.0), compared to African-Americans with MI due to HTN. Data were not sufficient to generate genotypic odds ratios of 1.5 or greater. These data firer suggest that the EDN-1 gene is significantly associated with hypertensive cardiomyopathy in African-Americans, i.e. abnormal activity of the EDN-1 gene predisposes African-Americans to hypertensive cardiomyopathy.

For Caucasians with hypertensive cardiomyopathy the odds ratio for the A allele was 2.5 (95% CI, 0.8-7.8), compared to Caucasians with MI due to HTN. Data were not sufficient to generate genotypic odds ratios of 1.5 or greater. These data further suggest that the EDN-1 gene is significantly associated with hypertensive cardiomyopathy in Caucasians, i.e. abnormal activity of the EDN-1 gene predisposes Caucasians to hypertensive cardiomyopathy.

For Caucasians with CVA due to NIDDM the odds ratio for the A allele was 3.0 (95% CI, 1-9.4), compared to Caucasians with NIDDM only. The odds ratio for the homozygote (A/A) was 0, while the odds ratio for the heterozygote (C/A) was 1.5 (95% CI, 0.3-7.2).These data suggest that the A allele acts in a manner in this patient population. These data further suggest that the EDN-1 gene is significantly associated with CVA due to NIDDM in Caucasians, i.e. abnormal activity of the EDN-1 gene predisposes Caucasians to CVA due to NIDDM.

For African-Americans with MI due to NIDDM the odds ratio for the A allele was 2.3 (95% CI, 0.8-7), compared to African-Americans with NIDDM only. Data were not sufficient to generate genotypic odds ratios of 1.5 or greater. These data further suggest that the EDN-1 gene is significantly associated with MI due to NIDDM in African-Americans, i.e. abnormal activity of the EDN-1 gene predisposes African-Americans to MI due to NIDDM.

For African-Americans with seizure disorder the odds ratio for the C allele was 1.8 (95% CI, 0.9-3.9). The odds ratio for the homozygote (C/C) was 3.0 (95% CI, 0.6-14.9), while the odds ratio for the heterozygote (C/A) was 1.9 (95% CI, 0.6-5.8).These data suggest that the C allele acts in a dominant manner in this patient population with a greater than additive effect of allele dosage [3>3.8=(1.9+1.9−1.0)]. (Goldstein et al., Monogr. Natl. Cancer Inst.; 26:49-54, 1999). These data further suggest that the EDN-1 gene is significantly associated with seizure disorder in African-Americans, i.e. abnormal activity of the EDN-1 gene predisposes African-Americans to seizure disorder.

For Caucasians with seizure disorder the odds ratio for the A allele was 1.7 (95% CI, 0.6-5.1). Data were not sufficient to generate genotypic odds ratios of 1.5 or greater. These data further suggest that the EDN-1 gene is significantly associated with seizure disorder in Caucasians, i.e. abnormal activity of the EDN-1 gene predisposes Caucasians to seizure disorder.

According to MatInspector (GENOMATIX; see above for URL and reference), the C2657→A SNP disrupts a binding site for CEBPB (CCAAT/enhancer binding protein-beta; Quandt K et al., Nucleic Acids Res., 23: 4878-4884 (1995)). CEBPB binds to a core sequence of four nucleotides, GMAA, in an overall sequence of 14 nucleotides (ref. Akira, S. et al. A nuclear factor for IL-6 expression (NF-IL6) is a member of a C/EBP family. EMBO J. 1990 Jun:9(6):1897-1906). CEBPB_{—}01 binding sites, which occur on average 2.07 times per 1000 base pairs of random genomic sequence in vertebrates, are predicted to occur at positions 2647 to 2660 on the (+) strand of reference sequence J05008.1 (matrix score 0.952, with 1.0 being an identical match), as well as from position 2670 to 2657 on the (−) strand (matrix score 0.891 out of a possible 1.0). In neither case, however, does the C2657→A SNP alter a nucleotide critical for binding.

TABLE 17

| | | | Reference | | |
|---|---|---|---|---|---|
| Gene | Region | Location | Type | Variant | SEQ ID |
| EDN-1 | Promoter | 2239 | T | G | 1 |
| | | 2657 | A | C | 1 |

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and fictions, but puts them forth only as possible explanations.

It is to be further understood that the specific embodiments of the present invention as set forth are not intended as~being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for diagnosing a genetic susceptibility for a disease, condition, or disorder in a subject comprising:
obtaining a biological sample containing nucleic acid from said subject; and analyzing said nucleic acid to detect the presence or absence of a single nucleotide polymorphism in the EDN-1 gene, wherein said single nucleotide polymorphism is associated with a genetic predisposition for a disease, condition or disorder selected from the group consisting of hypertension, end stage renal disease due to hypertension, non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infarction due to hypertension, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder.

2. The method of claim 1, wherein the gene EDN-1 comprises SEQ ID NO: 1.

3. The method of claim 1, wherein said nucleic acid is DNA, RNA, cDNA or mRNA.

4. The method of claim 2, wherein said single nucleotide polymorphism is located at position 2239 or 2657 of SEQ ID NO: 1.

5. The method of claim 4, wherein said single nucleotide polymorphism is selected from the group consisting of T2239→G and C2657→A and the complements thereof namely A2239→C and G2657→T.

6. The method of claim 1, wherein said analysis is accomplished by sequencing, mini sequencing, hybridization, restriction fragment analysis, oligonucleotide ligation assay or allele specific PCR.

7. An isolated polynucleotide comprising at least 10 contiguous nucleotides of SEQ ID NO: 1, or the complement thereof, and containing at least one single nucleotide polymorphism at position 2239 or 2657 of SEQ ID NO: 1 wherein said at least one single nucleotide polymorphism is associated with a disease, condition or disorder selected from the group consisting of hypertension, end stage renal disease due to hypertension, non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infarction due to hypertension, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder.

8. The isolated polynucleotide of claim 7, wherein at least one single nucleotide polymorphism is selected from the group consisting of T2239→G and C2657→A and the complements thereof namely A2239→C and G2657→T.

9. The isolated polynucleotide of claim 7, wherein said at least one single nucleotide polymorphism is located at the 3′ end of said nucleic acid sequence.

10. The isolated polynucleotide of claim 7, further comprising a detectable label.

11. The isolated nucleic acid sequence of claim 10, wherein said detectable label is selected from the group consisting of radionuclides, fluorophores or fluorochromes, peptides, enzymes, antigens, antibodies, vitamins or steroids.

12. A kit comprising at least one isolated polynucleotide of at least 10 contiguous nucleotides of SEQ ID NO: 1 or the complement thereof, and containing at least one single nucleotide polymorphism associated with a disease, condition, or disorder selected from the group consisting of hypertension, end stage renal disease due to hypertension, non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infarction due to hypertension, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder; and instructions for using said polynucleotide for detecting the presence or absence of said at least one single nucleotide polymorphism in said nucleic acid.

13. The kit of claim 12 wherein said at least one single nucleotide polymorphism is located at position 2239 or 2657 of SEQ ID NO: 1.

14. The kit of claim 13 wherein said at least one single nucleotide polymorphism selected from the group consisting of T2239→G and C2657→A and the complements thereof namely A2239→C and G2657→T.

15. The kit of claim 12, wherein said single nucleotide polymorphism is located at the 3′ end of said polynucleotide.

16. The kit of claim 12, wherein said polynucleotide further comprises at least one detectable label.

17. The kit of claim 16, wherein said label is chosen from the group consisting of radionuclides, fluorophores or fluorochromes, peptides enzymes, antigens, antibodies, vitamins or steroids.

18. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease, condition, or disorder selected from the group consisting of hypertension, end stage renal disease due to hypertension, non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infarction due to hypertension, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder; and instructions for using said polynucleotide for detecting the presence or absence of said single nucleotide polymorphism in a biological sample containing nucleic acid.

19. The kit of claim 18, wherein said single nucleotide polymorphism site is located at position 2239 or 2657 of SEQ ID NO: 1.

20. The kit of claim 19, wherein said at least one polynucleotide further comprises a detectable label.

21. The kit of claim 20, wherein said detectable label is chosen from the group consisting of radionuclides, fluorophores or fluorochromes, peptides, enzymes, antigens, antibodies, vitamins or steroids.

22. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with a disease, condition, or disorder selected from the group consisting of hypertension, end stage renal disease due to hypertension, non-insulin dependent diabetes mellitus, end stage renal disease due to non-insulin dependent diabetes mellitus, lung cancer, breast cancer, prostate cancer, colon cancer, atherosclerotic peripheral vascular disease due to hypertension, cerebrovascular accident due to hypertension, cataracts due to hypertension, cardiomyopathy with hypertension, myocardial infection due to hypertension, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder; and treating said subject for said disease, condition or disorder.

23. The method of claim 22 wherein said nucleic acid is selected from the group consisting of DNA, cDNA, RNA and mRNA.

24. The method of claim 22, wherein said at least one single nucleotide polymorphism is located at position 2239 or 2657 of SEQ ID NO: 1.

25. The method of claim 22 wherein said at least one single nucleotide polymorphism is selected from the group consisting of T2239→G and C2657→A and the complements thereof namely A2239→C and G2657→T.

26. The method of claim 22 wherein said treatment counteracts the effect of said at least one single nucleotide polymorphism detected.
